SEQUENCE LISTING (2 sequences)

SEQ ID NO: 1 - DNA, 26 bp, Artificial
  Synthesized DNA
taatacgactcactatagggagacaa

SEQ ID NO: 2 - DNA, 24 bp, Artificial
  Synthesized DNA
nttcgacaggaggctcacaacagg

(19) United States
(12) Patent Application Publication
(10) Pub. No.: US 2008/0003678 A1
(43) Pub. Date: Jan. 3, 2008

(54) Cell separation chip and cell culturing method using the same

(75) Inventors: Akihiro Hattori, Tokyo (JP); Hideyuki Terazono, Tokyo (JP); Kenji Yasuda, Tokyo (JP)

Correspondence Address: MORGAN LEWIS & BOCKIUS LLP, 1111 PENNSYLVANIA AVENUE NW, WASHINGTON, DC, 20004, US

(73) Assignee: On-chip Biotechnologies Co., Ltd.

(21) Appl. No.: 11/819,681

(22) Filed: Jun. 28, 2007

(30) Foreign Application Priority Data
Jun. 28, 2006 (JP) 2006-178649

(51) Int. Cl. C12N 5/06; C12M 1/00

(52) U.S. Cl. 435/383; 435/308.1

(57) ABSTRACT

An inexpensive cell analysis and separation apparatus using a flow channel formed on one surface of a substrate and a chip replaceable for each sample, and a method for culturing the separated cells without contamination, are provided. A flow channel for allowing a cell-containing buffer solution to flow is provided. Cells are detected in the middle of the flow channel, and separated to a plurality of downstream flow channels based on whether each cell fulfills a predetermined condition. A culturing tank for collecting the condition-fulfilling cells is covered with a semipermeable membrane at a top surface so as to prevent contamination during cell separation. When the cell separation is finished, the flow channel communicated with the culturing tank accommodating the condition-fulfilling cells is closed, and the culturing tank is cut off from the apparatus and put into a culturing device containing a predetermined medium to culture the cells.

### BACKGROUND OF THE INVENTION

1. Field of the Invention

The present invention relates to a cell separation and culturing apparatus and a cell culturing method using the same.

2. Description of the Related Art

In biological tissues of a multi-cell organism, various cells each play the respective role, so that the organism maintains various functions in harmony as a whole. When the cells are partially cancerated, (herein, the term “cancer” encompasses a tumor), such cells become neoplasm which is different from the surrounding area. A cancer area and a normal tissue area far from the cancer area are not necessarily distinguishable along a clear borderline, and the area surrounding the cancer area is somewhat influenced by the cancer area. Accordingly, in order to analyze functions of organ tissues, it is necessary to separate a small number of cells existing in a small area.

In the field of regenerative medicine, it is now attempted to separate stem cells from the tissue and then culture, differentiate and induce the stem cells to regenerate a target tissue and finally to regenerate a target organ.

In order to identify or separate cells, some distinguishing index is needed. In general, cells are classified by the following methods.

(1) Morphological cell classification by visual observation: For example, diagnosis of a bladder cancer or urethra cancer is given by examining heterotypic cells expressing in urine; or cancer diagnosis is given by examining heterotypic cells in blood or by performing cytodiagnosis of a tissue.

(2) Cell classification using cell surface antigen (marker) dyeing by a fluorescent antibody method: In general, a cell surface antigen called a CD marker is dyed with a fluorescent labeling antibody specific thereto. This is used for cell separation by a cell sorter or for cancer examination by a flow cytometer or tissue dyeing. These are widely used for cytophysiological studies or industrial use of cells as well as for medical studies.

(3) A target stem cell is separated as follows. Cells including the target stem cell are roughly separated using a fluorescent colorant in the cells as reporter, and then actually cultured. The cells need to be cultured since no effective marker for a stem cell has been established. Among the cultured cells, only the cell which is differentiated and induced is used as the target stem cell.

To separate and recover a specific cell in a culture solution as described above is an important technology in biological and medical analysis. When using the difference in the specific gravity of the cells for the separation, a velocity sedimentation method is usable. When there is almost no difference in the specific gravity among the cells, for example, when an unsensitized cell needs to be distinguished from a sensitized cell, it is necessary to separate the cells one by one based on the information obtained by dyeing the cells with a fluorescent antibody or information obtained by visual observation.

For such a technology, a cell sorter, for example, is usable. With a cell sorter, cells treated with a fluorescent dye are isolated one by one into a charged liquid droplet. While each liquid droplet is dropping, a high electric field is applied thereto in an optional direction in the normal plane with respect to the dropping direction based on presence/absence of fluorescence in the cell and the amount of scattered light, to control the dropping direction of the liquid droplets. Thus, the liquid droplets are fractionated and recovered into a plurality of vessels located below. (Non-patent document 1: Kamarck, M. E., Methods Enzymol. Vol. 151, p150-165 (1987))

This technology has problems that the apparatus is expensive and large-scaled, a high electric field of several thousand volts is necessary, a large number of samples are necessary, the cells may possibly be damaged while the liquid droplets are created, and the samples cannot be directly observed. In order to solve these problems, a cell sorter has recently been developed for creating microscopic flow channels using a microscopic processing technology and separating the cells flowing in a laminar flow in the flow channel while being directly observed with a microscope. (Non-patent document 2: Micro Total Analysis, 98, pp. 77-80 (Kluwer Academic Publishers, 1998); Analytical Chemistry, 70, pp. 1909-1915 (1998)) However, with the cell sorter for creating the flow channels using the microscopic processing technology, the speed of response to the observation means for sample separation is slow. For practical use, a processing method providing a higher responding speed without damaging the samples is necessary.

In order to solve these problems, the present inventors developed, using a microscopic processing technology, a cell analysis and separation apparatus capable of fractionating samples based on the microscopic structure and the fluorescent distribution in the samples, and analyzing and separating the cell samples in a simple manner without damaging the samples to be recovered, and filed a patent application (Japanese Laid-Open Patent Publication No. 2003-107099, Japanese Laid-Open Patent Publication No. 2004-85323, and WO2004/101731). This cell sorter is sufficiently practical on a laboratory level. For general-purpose uses, however, new technological development is necessary on the liquid transportation method, recovery method, and sample preparation.

[Non-patent document 1] Kamarck, M. E., Methods Enzymol. Vol. 151, p150-165 (1987)

[Non-patent document 2] Micro Total Analysis, 98, pp. 77-80 (Kluwer Academic Publishers, 1998); Analytical Chemistry, 70, pp. 1909-1915 (1998)

[Patent document 1] Japanese Laid-Open Patent Publication No. 2003-107099

[Patent document 2] Japanese Laid-Open Patent Publication No. 2004-85323

[Patent document 3] International Publication WO2004/101731 pamphlet

### SUMMARY OF THE INVENTION

The present invention has an object of establishing a cell separation and culturing chip and a cell separation and culturing method capable of detecting and separating a predetermined cell with certainty using a flow channel formed in one surface of a substrate; and providing a cell analysis, separation and culturing apparatus using a chip which is inexpensive and replaceable for each sample. The present invention has another object of providing a culturing method capable of transferring the separated cells to a culturing step without contaminating the cells.

When a liquid is caused to flow in a microscopic flow channel formed in one surface of a substrate, the liquid is generally made into a laminar flow. At a glance, it appears that there is no flow rate distribution in a cross-sectional direction of the flow channel. However, when a cell suspension is caused to flow in such a microscopic flow channel, the phenomenon that the cells contact the walls of the flow channel frequently occurs. The cells in contact with the walls receive a resistance against the flow and is decreased in the flow rate, and thus contact cells flown thereafter. When such a phenomenon occurs in a cell sorter or a flow cytometer, it becomes difficult to separate and detect the cells. In general, a sheath flow technology is used to prevent such a phenomenon. According to the sheath flow technology, a liquid flow flowing at a high rate is used as a sheath and a cell suspension is poured into a core thereof, so as to arrange the cells in one line. Then, the sheath flow and the core flow are merged, and the resultant flow is flushed into air as a jet flow. Since no wall is used with this conventional method, the cells can be separated in an ideal state without the phenomenon that the cells contact the walls.

However, it is very difficult to stably form a jet flow using a sheath. A practically usable apparatus is very expensive, and the cells for forming the sheath are not replaceable for each sample. Whether using a large-scale apparatus or a cell sorter formed on a chip, all the conventional methods other than the method disclosed by the present invention inventors need a pump for transporting a sample liquid and another pump for transporting a sheath liquid. These pumps are located separately from the chip, and need to be re-jointed each time the chip is replaced. In addition, each time the chip is replaced, the sample liquid transportation speed and the sheath. liquid transportation speed need to be adjusted to be balanced. For such precise controls, large-scale, highly stable pumps are required.

In the case where the cell sorter is structured on a chip, it is important that the liquid transportation part and the culturing tanks for separated cells should also be formed on the chip, so that all the elements other than the optical system should be provided on the chip. Such a closed structure improves ease of use and reduces the cost. Owing to the closed structure, which has all the elements on the chip other than the optical system, the cell sorter chip is used in a new manner, i.e., is disposed after used for each sample. For example, when stem cells are separated or used for clinical testing, means for preventing the stem cells from being contaminated with cells derived from the other sample tissue is indispensable. Where each cell sorter chip is disposed after single use, the means for preventing contamination is not necessary. By forming an important part of the cell sorter as a chip, the apparatus is reduced in size and cost. Because the chip is replaced for each sample, cross-contamination is completely prevented. An object of the present invention is to construct such a cell separation and culturing system with no cross-contamination, which is indispensable in the field of medicine, especially in the field of regenerative medicine, and to use this system to provide a cell separation and culturing method with no contamination using this system.

One important technological element for realizing a chip-type cell sorter is a mechanism for separating cells flowing in the microscopic flow channel. Various types of separation mechanisms have been proposed. For example, for moving cells in an intended direction, mechanisms using ultrasonic, magnetic field, flow channel switching by a valve, laser tweezer, high frequency electric field, and DC electric field have been proposed. A method of using a low voltage DC electric field works well with high reproducibility without damaging the cells and without using any special apparatus. However, when a general metal electrode is used for separating the cells at high speed using a DC electric field, there occurs a problem that the buffer solution is electrolyzed and the apparatus cannot be used stably for a long period of time. A method having a highest possibility of being practically usable is described in WO2004/101731.

With the mass-produceable cell sorter chip created using the microscopic processing technology described in WO2004/101731, a DC electric field is applied to the flow channel in which the cells are flowing using gel electrodes as a mechanism for separating the cells, and thus the cells are separated in an electrophoretic manner. This arrangement is proposed in order to alleviate the influence of electrolysis to some extent. For the gel electrodes, an electrolyte-containing agarose gel or the like is used. Since only the gel in a small hole is existent in the vicinity of the flow channel in which the cells are flowing, the influence of bubble generation can be prevented for a certain period of time but not a sufficient length of time. There is another problem that the gel needs to be supplied and stored in the state of containing an electrolyte buffer solution, and thus is vulnerable to drying and is not suitable for long-time storage. The gel is destroyed by freezing harm and thus cannot be frozen for long-time storage.

Therefore, it is useful to provide a cell sorter chip disposable for each sample in a true sense using gel electrodes which prevent the generation of bubbles while being supplied with an electric field and are durable against drying and freezing while being stored.

For separating cells using a flow channel formed in one surface of a substrate, it is necessary to provide a site for cell recognition in a specified area in the flow channel and also provide an algorithm for recognizing cells by some means. For using the cell sorter for cell separation, it is necessary to provide a separation section downstream with respect to the cell detection section. In general, there are the following three methods for cell detection.

(1) Laser light or the like is radiated to a detection section on the flow channel. Light which is scattered when a cell passes the detection section is detected; or when the cell is colored with a fluorescent dye, the fluorescence is detected.

(2) An electrode is provided on a detection section. The impedance or conductance change which is caused when a cell passes the electrode is detected.

(3) A cell is detected as an image using a CCD camera or the like.

With the method of (1), the cell recognition is performed substantially at one point, and thus high speed processing is possible even when the cells continuously flow at high rate. Therefore, the method of (1) is used for a large-scale cell sorter using the technology of encapsulating the cells in liquid droplets and thus moving the cells between the detection section and the separation section at a constant speed.

With the method of (2) also, high speed processing is possible. However, the method of (2) is generally adopted for a flow cytometer used for cell classification because the moving velocity of the cells after the detection cannot be measured and it is difficult to combine this method with a fractionation mechanism.

The method of (3) appears to be simple, but is not generally used. The reason is that a plurality of cells constantly moving in the flow channel need to be handled and thus the load on the cell sorter for image processing is large.

For performing similar cell recognition and then cell separation in a flow channel incorporated in a small area of one surface of a substrate, various other problems occur. First, the moving velocity of the cells flowing in the flow channel is not the same for all the cells but varies in accordance with the factors such as, for example, the shape and size of the cell and whether the cell is flowing in the center of the flow or close to the wall. As a result, especially the time between the cell recognition and the cell separation performed downstream with respect to the site of cell recognition is varied. Due to the difference in moving velocity of the cells, one cell may occasionally go past another cell in the flow channel. This is a problem to be addressed for separating the cells with certainty by the method (1) or (2), by which the cell is observed at one point. In addition, an algorithm for recognizing the cells flowing in the flow channel continuously at high rate by detecting the cells as images using a CCD camera or the like and choosing necessary cells is required.

As described above, as a specific architecture for constructing a cell separation and culturing apparatus or a flow cytometer on a chip, the present invention especially proposes a shape of cell flow channel, a structure of a cell suspension transportation part, a structure of an electrode part durable against the long-time storage to be provided in the flow channel, a separation algorithm, and a cell measurement, separation and culturing chip for cells from a tissue fraction or a cell mass as a sample.

The present invention also proposes a culturing method capable of transferring the cells collected by the separation and culturing chip to a culturing step without contaminating the cells.

Cells assumed in the present invention range from small cells such as bacteria to large cells such as animal cells, for example, cancer cells. Accordingly, the cell size assumed in the present invention is from about 0.5 μm to about 30 μm. For separating cells using a flow channel incorporated in one surface of a substrate, a first issue to address is the width of the flow channel (shape of the cross-section). The flow channel is formed substantially two-dimensionally in one surface of the substrate, in a space of about 10 to 100 μm in the thickness direction of the substrate. Based on the size of the cells, a space of 5 to 10 μm is suitable for bacteria, and a space of 10 to 50 μm is suitable for animal cells. As described above, it is necessary to prevent the cells from contacting the walls, first of all. The cells are prevented from contacting the walls by injecting another liquid from both sides of the flow channel as side flows in which the cell suspension flows. As a result of studying a method of merging liquids, it was found that the largest effect is obtained when the width (substantially, the cross-sectional area) of the pre-merging flow channel in which the cell suspension flows is equal to the width of the post-merging flow channel and the lengths of the side flows to be merged are equal to each other. When the width of the post-merging flow channel is too large, the effect of keeping the cells far from the walls is reduced. When the width of the post-merging flow channel is too small, the flow rate of the cell flow is too high after the merging and it becomes difficult to detect the cells, and the frequency at which the cells appear is significantly reduced. When the lengths of the two side flows are different, the flow resistances are different. As a result, the central flow channel in which the cells flow becomes closer to one of the side flows.

For constructing a cell separation and culturing apparatus or a flow cytometer on a chip, it is very difficult to control the flow rates of the cell suspension and the side flows. A large-scale non-pulsation pump capable of stably providing a flow rate of several tens of picoliters per minute would solve these problems. When a disposable chip is used, however, the chip and the pump need to be jointed each time and this presents a problem in terms of reproduceability and ease of use. It has been attempted to incorporate a pump on a chip. The present invention, by contrast, solves these problems not by using any pump and but by utilizing free fall of liquid. Practically, a reservoir is provided at an entrance and an exit of the flow channel, and the surface level in the exit-side reservoir is made lower than that of the entrance-side reservoir. In this manner, a microscopic amount of liquid can be sent with no pulsation.

For transporting a plurality of liquid flows in such a system using the difference in liquid surface level, it is difficult to control the flow rates of the plurality of liquid flows. Even a slight difference in the surface level among the plurality of liquid flows varies the flow ratio of the cell suspension flow with respect to the side flows. The present invention solves this problem by integrating a reservoir for accommodating the cell suspension and a reservoir for accommodating the buffer solution forming the side flows so as to precisely match the surface level in the reservoirs. Namely, the difference between the surface level on the entrance side and the surface level on the exit side is used as a driving force to transport the liquids, so that the flow ratio of the cell suspension flow with respect to the side flows is not varied. As a reservoir having such a structure, the present invention proposes a reservoir which is divided into two parts by a partition, in which the bottom of one part is in communication with a flow channel for samples and the bottom of the other part is in communication with flow channels for side flows. Substantially, the surface levels of the two parts are matched by the principle of a siphon. Above the partition, the different types of liquids are in communication with each other, but the cells have a higher specific gravity than that of the buffer solution and therefore the cells never go over the partition to flow into the side flows.

Alternatively, the space above the liquid surface in the reservoir may be used as a closed space and pressurized by air in order to increase the driving force.

In the cell separation and culturing chip according to the present invention, the cell detection section is located in a part where the side flows are merged with the cell suspension flow. For capturing a cell in an image for evaluation, an area is provided in which the post-merging flow channel can be observed with a CCD camera. Optionally, a cell separation area is provided downstream with respect to the observation area. Instead of using an image, cells flowing down the flow channel may be irradiated with laser light or the like and the light scattered by each cell when the cell passes the cell detection area may be detected by a light detector. Alternatively, in the case where the cells are modified with fluorescence, the fluorescence may be detected by a light detector. In these cases also, the cell separation area is provided downstream with respect to the cell detection area.

At the entrance of the cell separation area (i.e., separation section), a flow channel having only a buffer solution (or a medium) is merged as a flow channel for moving the cells, and is branched downstream with respect to the cell separation area. The cells are separated in the cell separation section as follows. Electrodes are provided as means for externally pressurizing and moving the cells in the cell separation area, and a flow channel for discharging the separated cells is provided. For charging the flow channel of the cells by applying a voltage to the electrode and thus applying ions, the cells are moved in a direction of a synthetic vector of the ion flowing direction and the liquid flowing direction in the flow channel. The cells, which are charged negative, are moved toward a positive electrode. A negative electrode is located downstream and a positive electrode is located upstream in the direction of the post-merging flow channel, so as to control the synthetic vector for moving the cells and change the flow channel of the cells at a small amount of electric current. The cells, the flow channel of which is to be changed, and the remaining cells are moved to different flow channels and thus separated from each other.

The flow channel for the samples is already merged upstream with side flows upstream with respect to the cell separation area. By contrast, the flow channel for only the buffer solution (or the medium) is not merged with any other flow upstream with respect to the cell separation area. The sample flow channel, and buffer solution flow channel, and the post-merging flow channel have an equal width. In the post-merging flow channel, the rate of sample flow is faster because of the side flows. Therefore, in the separation section, the sample flow is slightly deflected toward the flow from the buffer solution flow. This is important to provide an effect of easily allowing the cells to be moved from the sample flow to the buffer solution flow. When no electric current is provided, the cells flow in the center of the sample flow and continue to flow without changing the channel.

The electrodes located in the separation section each include a metal portion, which contacts a space filled with gel via a liquid junction (a narrow tube containing a liquid filling the space; the liquid is gel in this example). Herein, these electrodes will be referred to as “gel electrodes”. The negative gel electrode is formed of a gel matrix containing a buffer solution having a low pH value as a result of absorbing generated hydroxy ions, and the positive gel electrode is formed of a gel matrix containing a buffer solution having a high pH value as a result of absorbing generated hydrogen ions. For the gel matrix, a gel assuming a mesh structure which is generally used in biochemistry, for example, agarose or polyacrylamide is usable. Owing to such a structure, gas generation caused by electrolysis of the gel electrodes is suppressed and the cell analysis and separation can be performed stably. Since the metal portions of the electrodes do not directly contact the cells, the cells are not damaged by the electrode surfaces. The cell samples are prevented from being damaged and are also prevented from being lost due to the electrolysis of the electrodes.

Gel electrodes are significantly advantageous over the metal electrodes described in Japanese Laid-Open Patent Publication No. 2003-107099, but has a problem when providing a chip to the user. Gel electrodes are wet-type electrodes and therefore instable during storage. This practically requires the user to fill the chip with the gel immediately before the use. According to the present invention, the gel is made storable at room temperature for a certain time period by adding trehalose or other nonreducible disaccharide, glycerol, ethyleneglycol or the like. In addition, it is usually difficult to store gel in a frozen state because the gel structure is destroyed when being frozen. According to the present invention, gel is rapidly frozen by addition of trehalose or the like. In this manner, expression of the ice crystals which destroy the gel structure can be suppressed, which enables the gel electrodes to be stored in a frozen state for a long period of time.

In addition, the cell separation and culturing apparatus according to the present invention may include means for capturing impurities at an upstream position in the flow channel at which the fluid, containing the sample to be introduced to the cell separation area, is introduced and thus preventing the flow channel from clogging.

Namely, the present invention is directed to a cell separation and culturing apparatus comprising a cell separation space, at least one flow channel for injecting a cell-containing fluid to the cell separation space, at least two flow channels for discharging the fluid from the at least one flow channel, and means for applying an external force to a cell in the cell separation area. The flow channels are located such that in accordance with whether the external force is applied to the cell separation area or not, the cells are discharged to different flow channels from the cell separation area. When a cell is evaluated as fulfilling a predetermined condition and is discharged to the selected flow channel from the cell separation area, this cell reaches a culturing tank located at the end of the selected flow channel. When a cell is evaluated as not fulfilling the predetermined condition and is discharged to the non-selected flow channel from the cell separation area, this cell reaches a culturing tank located at the end of the non-selected flow channel. These culturing tanks are provided in a common exit-side reservoir. In the case where the cells flowing in the non-selected flow channel do not need to be cultured, the culturing tank at the end of the non-selected flow channel may be omitted. The culturing tanks are covered with a semipermeable membrane at a top surface thereof, and therefore are protected against bacteria or the like. As the separation proceeds, the culturing tanks are filled with the buffer solution (medium) accumulated in the exit-side reservoir. Owing to such an arrangement, the cells in the culturing tanks can be cultured as time passes by merely being left in the state when the separation operation is finished. In addition, the culturing tank accommodating the cells can be cut off from the cell separation and culturing apparatus and put into a culturing device having an appropriate medium. In this case, the cells can be cultured by the medium introduced to the culturing tank via the semipermeable membrane.

With this cell separation and culturing apparatus, an external force is applied to the cells in the cell separation area. Therefore, the electrodes or the like do not directly contact the cell-containing buffer solution. Since the cells are separated by providing an electric current (i.e., ions) at a low voltage, the cells are not heavily damaged.

The cell recognition and separation algorithm has the following features.

For capturing a cell as an image for evaluation, an area is provided in which the post-merging flow channel can be observed with a CCD camera. The measuring range is expanded two-dimensionally to identify and trace the cell by image recognition. Thus, cell separation is performed with certainty. The important element at this point is the image capturing rate. With a general camera with a video rate of 30 frames/sec., all the cells cannot be imaged. A video rate of at least 200 frames/sec. is required to recognize the cells flowing in the flow channel at high rate.

The image processing method will be described. Because the capturing rate is high, very complicated image processing cannot be performed. Regarding image recognition, the cell moving velocity varies depending on each cell, and one cell may go past another cell as described above. Therefore, when each cell appears in the image frame for the first time, the cell is numbered. The same cell is managed with the same number until the cell disappears from the image frame. In this manner, how an image of each cell is moved in a plurality of continuous frames is managed with the number. The cell in one frame and the same cell in another frame are linked with the condition that a cell are moved from an upstream position to a downstream position in each frame and that the moving velocity of a specified numbered cell recognized in the image is within a certain range. Thus, even if one cell goes past another cell, each cell can be traced with certainty.

In this manner, the cell recognition is realized. The cells are numbered as follows. A cell image is binarized, and the center of gravity thereof is obtained. The luminance center of gravity, area size, circumferential length, longer diameter and shorter diameter of the binarized cell are obtained, and the cell is numbered using these parameters. At this point, each cell image is automatically stored as an image because it is beneficial to the user.

In cell separation, only specific cells among the numbered cells need to be separated. The index for separation may be the information on the above-mentioned luminance center of gravity, area size, circumferential length, longer diameter, shorter diameter or the like, or information obtained by fluorescence detection performed in addition to the image capturing. In any way, the cells detected in the cell detection area are separated in accordance with the numbers. Practically, the moving velocity (V) of each numbered cell is calculated from the images taken at an interval of a predetermined time period. A voltage is applied to a cell of a target number when such a cell is between the electrodes at a timing of (L/) to (L/+T), where L is the distance from the cell detection area to the cell separation area and T is the application period. In this manner, the cells are electrically separated.

The present invention realizes a disposable cell separation and culturing chip capable of stably separating cells, and cell culturing with no contamination.

### BRIEF DESCRIPTION OF THE DRAWINGS

FIG. 1 is a plan view schematically showing one example of a system structure of a cell separation and culturing apparatus according to the present invention;

FIG. 2 is a view illustrating the manner in which a cell-containing buffer solution in a microscopic flow channel 204 is merged with a buffer solution in microscopic flow channels 205 and 205′ to flow down a microscopic flow channel 240, and is further merged with a buffer solution in a microscopic flow channel 204′ immediately before a cell detection area 221 to flow down a microscopic flow channel 247;

FIG. 3 is a view illustrating the cell distribution in the post-merging flow channel 240 as a result of the buffer solution flowing down the flow channel 204 being pushed to the center of the flow channel 240 by the buffer solution flowing down the flow channels 205 and 205′;

FIG. 4(A) and FIG. 4(B) are cross-sectional views showing a problem caused by increasing the width of a flow channel and an example of means for solving the problem;

FIG. 5(A) is a cross-sectional view taken along line (A)-(A) in FIG. 1 and seen in the direction of the arrows thereof for illustrating a reservoir 203, openings 201 and 201′, and the flow channels 204 and 204′ described with reference to FIG. 4(A) and FIG. 4(B) in more detail; and FIG. 5(B) is a cross-sectional view taken along line (B)-(B) in FIG. 1 and seen in the direction of the arrows thereof for illustrating culturing tanks 213 and 214, openings 211 and 212, flow channels 218 and 219, a semipermeable membrane 280, and a reservoir 285 on the flow channel exit side in more detail;

FIG. 6 is a plan view showing a system structure of a cell separation and culturing apparatus with a different structure of gel electrode section;

FIG. 7 illustrates an algorithm for recognizing cells from an image captured by a CCD camera and numbering and identifying each of the cells;

FIG. 8 is a plan view schematically showing one example of a system structure of a cell separation and culturing apparatus, which has a special arrangement for the introduction of sample cells as compared to the structure shown in FIG. 1;

FIG. 9(A), FIG. 9(B) and FIG. 9(C) are partial cross-sectional views illustrating the arrangement of the sample cell introduction section in FIG. 8;

FIG. 10 illustrates a flow of processing for specifically labeling cell surface antigen CD4-presenting cells with a P-phycoerythrin-modified RNA aptamer and separating the cells by a cell separation and culturing apparatus;

FIG. 11 shows an examination result of the influence on the fluorescence intensity of β-phycoerythrin as an identifying substance exerted by addition of nuclease;

FIG. 12 shows that the cell surface antigen CD4-presenting cells obtained by removing the β-phycoerythrin-modified RNA aptamer are culturable;

FIG. 13 is a view schematically showing an example of a cell culturing device for culturing the cells collected in the culturing tanks 213 and 214;

FIG. 14(A) through FIG. 14(C) are views showing an overview of processing for cutting off the culturing tanks 213 and 214 together with the chip substrate from the cell separation and culturing apparatus; and

FIG. 15 is an overall conceptual view of an optical system in the cell detection area 221.

### DESCRIPTION OF THE PREFERRED EMBODIMENTS

### (System Structure of the Cell Separation and Culturing Apparatus)

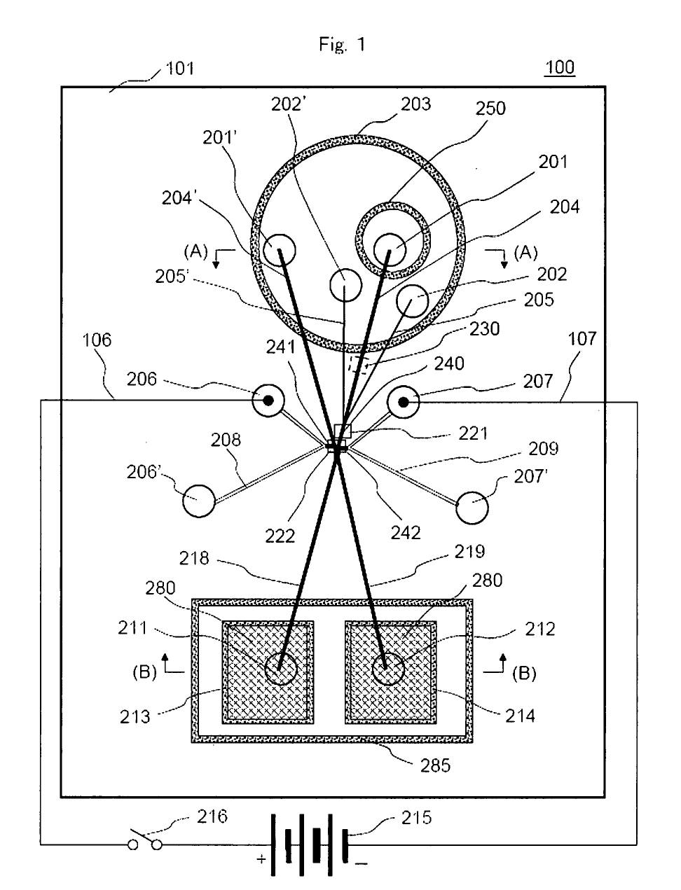

FIG. 1 is a plan view schematically showing one example of a system structure of a cell separation and culturing apparatus according to the present invention. A cell separation and culturing apparatus 100 includes a chip substrate 101. The cell separation and culturing apparatus 100 includes flow channels formed in a bottom surface thereof and openings communicating to the flow channels in a top surface thereof. The openings act as supply openings for samples and necessary buffer solutions (mediums). The cell separation and culturing apparatus 100 also includes reservoirs for supplying a sufficient amount of buffer solution and adjusting the flow rate in each flow channel. The flow channels can be formed by so-called injection molding, by which a plastic material such as PMMA or the like is injected into a mold. The chip substrate 101 has an overall size of 20×30×1 mm (t). In order to shape the grooves or through-holes formed in the bottom surface of the chip substrate 101 into flow channels or wells, a 0.1 mm thick laminate film is attached to the bottom surface having the grooves by thermal pressurization. Using an objective lens having a numerical aperture of 1.4 and a magnification of ×100, cells flowing in the flow channels can be observed through the 0.1 mm thick laminate film. In the case where the plastic material is highly light-transmissive, such cells can be observed also from above the chip substrate 101.

In the top surface of the chip substrate 101, holes 201 for introducing a cell-containing sample buffer solution to the microscopic flow channels, holes 201′, 202 and 202′ for introducing a buffer solution not containing cells, and a reservoir 203 for surrounding the holes 201, 201′, 202 and 202′, are formed. Accordingly, when a sufficient amount of buffer solution is supplied to the reservoir 203, the holes 201, 201′, 202 and 202′ are communicated with one another via the buffer solution. Thus, flow channels 204 and 204′ respectively communicated with the holes 201 and 201′ are supplied with the buffer solution at an equal level of liquid surface. Therefore, where the flow channels 204 and 204′ have an equal width (when having an equal height), or have a substantially equal cross-sectional area or length, the flow channels 204 and 204′ can provide substantially the same flow rate. Similarly, flow channels 205 and 205′ respectively communicated with the holes 202 and 202′ are supplied with the buffer solution at an equal level of liquid surface, and the flow rate of the buffer solution flowing in the flow channels 205 and 205′ can be adjusted to be a predetermined ratio to the flow rate of the buffer solution in the flow channel 204.

Around the hole 201 for introducing the cell-containing buffer solution, a wall 250 is provided for preventing the cell-containing buffer solution from diffusing. The wall 250 is lower than the wall of the reservoir 203, and the reservoir 203 is filled with the buffer solution up to a level higher than the wall 205.

The cell-containing buffer solution introduced to the hole 201 flows in the microscopic flow channel 204 (width: 20 μm, depth: 15 μm) and is introduced to a cell detection area 221 and a cell separation area 222. In the microscopic flow channel 204, a filter 230 directly built in the chip as a microscopic element is optionally provided in order to prevent the microscopic flow channel 204 from clogging. Meanwhile, the buffer solution not containing cells which is introduced to the holes 202 and 202′ flows in the flow channels 205 and 205′ (width: 12 μm; depth: 15 μm) and is merged with the cell-containing buffer solution in the microscopic flow channel 204. Reference numeral 240 represents a microscopic flow channel formed by merging the buffer solutions, which is introduced to the cell detection area 221. The microscopic flow channel 240 is further introduced to the cell separation area 222.

The buffer solution not containing cells which is introduced to the hole 201′ flows in the microscopic flow channel 204′ (width: 20 μm; depth: 15 μm) and is introduced to the cell separation area 222 to be merged with the microscopic channel 240. The width of the post-merging flow channel will be described later. The post-merging flow channel is separated at an exit of the cell separation area 222 into a microscopic flow channel 218 (width: 20 μm; depth: 15 μm) and a microscopic flow channel 219 (width: 20 μm; depth: 15 μm).

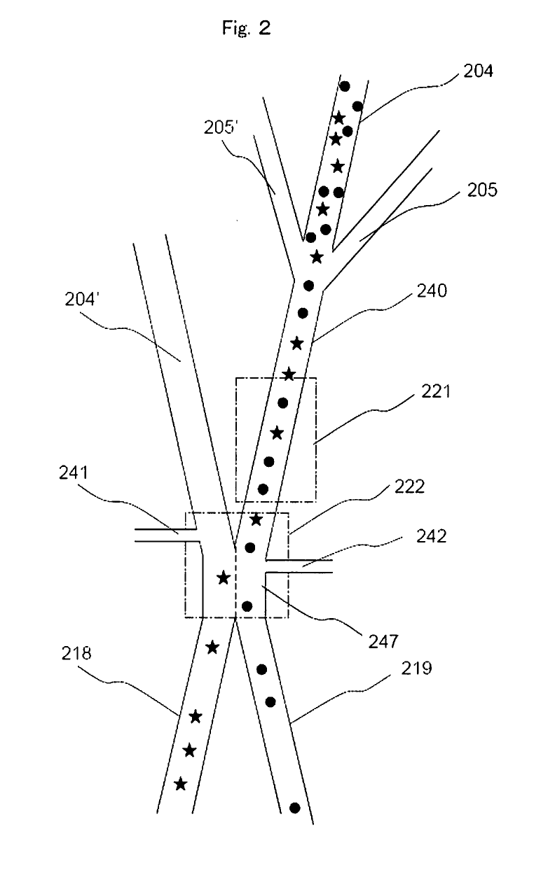

Reference numerals 206, 206′, 207 and 207′ represent holes for introducing an electrolyte-containing gel. The gel introduced to the holes 206 and 207 are respectively sent to the holes 206′ and 207′ via microscopic elements 208 and 209 (each is a bent groove of 200 μm (width)×15 μm (height)) which are formed in the bottom surface of the chip substrate 101. Therefore, the microscopic elements 208 and 209 are filled with the electrolyte-containing gel. Connection sections 241 and 242 are liquid junctions formed between the bent portions of the microscopic elements 208 and 209, and the microscopic flow channels 204 and 204′. The connection sections 241 and 242 each have a length of about 20 μm. Owing to this, in the cell separation area 222, the gel can be in direct contact with the buffer solution flowing in a flow channel 247 (FIG. 2) formed by merging the microscopic flow channels 240 and 204′. The gel and the buffer solution are in contact with each other in an area of about 15 μm (length along the flow channel)×15 μm (height). The holes 206 and 207 for introducing the gel each have an electrode represented with the black circle inserted therein. These electrodes are connected to a power supply 215 and a switch 216 via lines 106 and 107. The switch 216 is turned on only for applying a voltage to the buffer solution flowing in the flow channel 247 formed by merging the microscopic flow channels 240 and 204′.

The connection sections 241 and 242 which allow the gel to contact the buffer solution flowing in the flow channel 247 in the cell separation area 222 are structured such that the connection section 241 is located upstream with respect to the connection section 242 as shown in FIG. 1. Owing to this structure, when the electrode in the hole 206 is supplied with a positive voltage and the electrode in the hole 207 is supplied with a negative voltage, the cells flowing in the microscopic flow channel 240 can be efficiently moved to the flow channel 218. The reason is that when an electric current flows, an electrophoretic force acts on the cells charged negative, and this force and a vector received from the buffer solution are combined to form a synthetic vector. As result, in the structure of FIG. 1, as compared with the case where the connection sections 241 and 242 are located at the same position with respect to the flow (located symmetrically with respect to the flow line), the electric field is usable more efficiently and thus cells can be moved to the microscopic flow channel 218 or 219 more stably at a lower voltage.

Recovery holes 211 and 212 for recovering the cells separated in the cell separation area 222 are respectively formed downstream with respect to the microscopic flow channels 218 and 219. Culturing tanks 213 and 214 for accommodating the recovered cells are respectively provided around the holes 211 and 212. The culturing tanks 213 and 214 are surrounded by a reservoir 285. The reservoir 285 is located at an exit of the above-mentioned flow channels. The reservoir 285 is filled with the buffer solution to some level by the introduction thereof before the separation, but this level is lower than the level of the buffer solution in the reservoir 203 on the entrance side of the flow channels.

The level of the buffer solution in the reservoir 203 is higher than that in the reservoir 285. This level difference is used as a driving force for moving the buffer solution flowing in the flow channels and creates a stable flow with no pulsation. As long as a sufficient amount of buffer solution is accumulated in the reservoir 285, the cell-containing buffer solution introduced to the hole 201 can entirely flow to the flow channel 204. By putting a lid on the reservoir 203 to pressurize the space with air, the driving force for moving the buffer solution can be increased to raise the throughput.

The cells determined to fulfill a predetermined condition in the cell detection area 221 are separated from the other cells in the cell separation area 222 and collected in the culturing tank 213 after flowing down the flow channel 218. The cells determined not to fulfill the predetermined condition are separated in the cell separation area 222 and collected in the culturing tank 214 after flowing down the flow channel 219. The culturing tanks 213 and 214 are covered with a semipermeable membrane 280 at a top surface thereof in order to prevent the culturing tanks 213 and 214 from being contaminated with foreign substances during the cell separation. During the cell separation operation, the semipermeable membrane 280 is provided for protecting the culturing tanks 213 and 214 from the contamination. During the cell culturing operation performed in a culturing device after the flow channels 218 and 219 communicating with the culturing tanks 213 and 214 are closed and the culturing tanks 213 and 214 are cut off from the cell separation and culturing apparatus 100, the semipermeable membrane 280 acts as a membrane for supplying the cells with a medium as described later. When the cells not fulfilling the predetermined condition do not need to be cultured, the culturing tank 214 may be omitted.

FIG. 2 illustrates the manner in which the cell-containing buffer solution in the microscopic flow channel 204 is merged with the buffer solution in the microscopic flow channels 205 and 205′ and flows in the obtained microscopic flow channel 240 to reach the cell detection area 221, and is further merged with the buffer solution in the microscopic flow channel 204′ and flows in the obtained microscopic flow channel 247 to reach the cell separation area 222.

Now, the reason why the buffer solution not containing the cells which flows in the flow channels 205 and 205′ is merged with the cell-containing buffer solution flowing in the microscopic flow channel 204 at an upstream position with respect to the cell detection area 221 will be described. As described above, the flow channel 204 in which the cell-containing buffer solution flows is merged with the flow channels 205 and 205′ in which the buffer solution not containing the cells flows at an upstream position with respect to the cell detection area 221. The holes 201, 202 and 202′ provided at upstream ends of the flow channels are in the common reservoir 203 having a uniform liquid level. Because the flow channels 204, 205 and 205′ have an equal height, the flow rate of the buffer solution flowing in each of the flow channels 204, 205 and 205′ is in proportion to the width thereof. The width of the post-merging flow channel 240 is made substantially equal to that of the flow channel 204 for the cell-containing buffer solution. The term “substantially equal” means being equal in consideration of processing errors, and does not mean being strictly equal. Owing to this structure, the buffer solution flowing from the flow channel 204 is pushed to the center of the flow channel 240 at a constant ratio by the buffer solution flowing in the flow channels 205 and 205′. As a result, the cells, which flow in the flow channel 204 in contact with the side walls thereof, do not contact the side walls of the post-merging flow channel 240.

In the microscopic flow channel 247 in the cell separation area 222, the buffer solution from the flow channel 240 and the buffer solution from the flow channel 204′ flow while keeping the layers thereof, i.e., as if keeping the widths thereof, as represented with the dashed line, and flow down the flow channels 218 and 219. In the cell detection area 221, the cells fulfilling the predetermined condition are detected in the flow channel 240 and separated in the cell separation area 222 by an electric field acting by the function of the connection sections 241 and 242 in which the gel contacts the buffer solution flowing in the flow channels. Namely, when the electric field does not act, the cell-containing buffer solution flowing in the flow channel 240 flows down the flow channel 219. By contrast, when the electric field acts in the cell separation area 222, the cells in this location are pushed to the buffer solution flowing down the flow channel 218. In FIG. 2, the black circles represent the cells which do not fulfill the predetermined condition, and the stars represent the cells which fulfill the predetermined condition.

In FIG. 1 and FIG. 2, there are two routes, i.e., a route for cells selected as fulfilling the predetermined condition, and a route for non-selected cells. Alternatively, there may be three or more routes. In this case, flow channels are provided downstream with respect to the cell separation area 222, in addition to the flow channels 218 and 219 shown in FIG. 2. The number of the additional flow channels depends on the number of the routes. Cell information obtained in the cell separation area 222 is evaluated in accordance with the criterion prepared for the route determination, and the level of electric field to act on the cells by the connection sections 241 and 242 in the cell separation area 222 is controlled in accordance with the evaluation result. In consequence, the cells are controlled while flowing in the cell separation area 222 to reach the entrance of the route which is determined in accordance with the level of electric field and flow down the determined route. In this case, an independent culturing tank is provided at a downstream end of each of the additional route, needless to say.

Alternatively, a plurality of stages of cell separation areas may be provided in cascades in the route for the non-selected cells (flow channel 219) downstream with respect to the cell separation area 222. The route is separated into two at each stage, so that the cells in the flow channel 219 can be moved to the route for the selected cells (flow channel 218) at each stage. At which stage the cells are to be moved to the route for the selected cells is controlled in accordance with the cell information obtained in the cell detection area 221. In this case, a flow channel corresponding to the flow channel 204′ needs to be provided in each stage, and the structure is complicated.

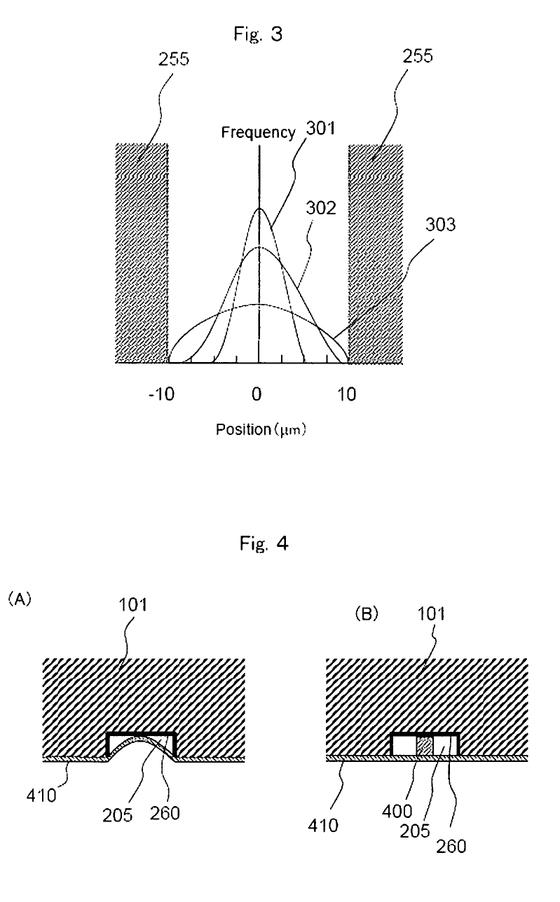

FIG. 3 illustrates the cell distribution in the post-merging flow channel 240 after the buffer solution flowing down the flow channel 204 is pushed to the center of the flow channel 240 by the buffer solution flowing down the flow channels 205 and 205′ at an upstream position with respect to the cell detection area 221. In FIG. 3, reference numeral 255 represents side walls of the flow channel. FIG. 3 shows the manner in which the cell-containing buffer solution flowing down the flow channel 204 having a width of 20 μm is pushed to the center of the flow channel 240 having a width of 20 μm by the buffer solution flowing down the flow channels 205 and 205′ each having a width of 12 μm. The horizontal axis represents the position in the flow channel 204, and the vertical axis represents the appearing frequency of the cells. Curve 301 indicates that in the case where the amount of the buffer solution flowing down each of the flow channels 205 and 205′ is about half of the amount of the buffer solution flowing down the flow channel 204, i.e., in the case where the width of each of the flow channels 205 and 205′ is about half of the width of the flow channel 204, the cells are distributed in a central area, having a width of an about 10 μm, of the flow channel 240. Curve 302 shows the cell distribution in the case where the flow channels 205 and 205′ have a smaller width, and curve 303 shows the cell distribution in the case where the flow channels 205 and 205′ are not provided. As is clear from curve 301, by appropriately setting the width of the flow channels 205 and 205′, the cells can be substantially allowed to flow with a certain distance from the side walls of the flow channels and thus can be substantially prevented from contacting the side walls.

Although not described with reference to FIG. 3, the flow channels 240 and 204′ are merged together in the cell separation area 222. Therefore, the characteristic shown in FIG. 3 tends to be slightly expanded toward the flow channel 204′ side, but no significant change occurs because the buffer solution in the flow channel 240 and the buffer solution in the flow channel 204′ substantially maintain the respective layers as represented with the dashed line in FIG. 2. Namely, the width of a part of the flow channel 247 corresponding to the flow channel 240 tends to be slightly expanded in the cell separation area 222 but there is no substantial change.

In FIG. 1, the flow channels 204, 204′, 205 and 205′ each have the same width throughout the length thereof. In order to decrease the resistance in the flow channel, the width of the flow channel may be expanded in the vicinity of the reservoir 203. In order to obtain an appropriate cell distribution, it is sufficient that a flow channel has a predetermined width over a length of, for example, about 100 μm. Therefore, the area having a larger width may be extended in the length direction in consideration of the resistance in the flow channel. For example, the flow channel 205 which forms a side flow for the buffer solution with no cells may be made wider than the flow channel 204 for the cell-containing buffer solution, and a portion of the flow channel 205 having the predetermined width may be shortened. In this case, the resistance in the flow channel 205 is smaller than that of the flow channel 204. As a result, the buffer solution from the flow channel 204 is pushed more to the center of the post-merging flow channel 240. Namely, cell distribution curve in FIG. 3 is made more acute as shown by curve 301.

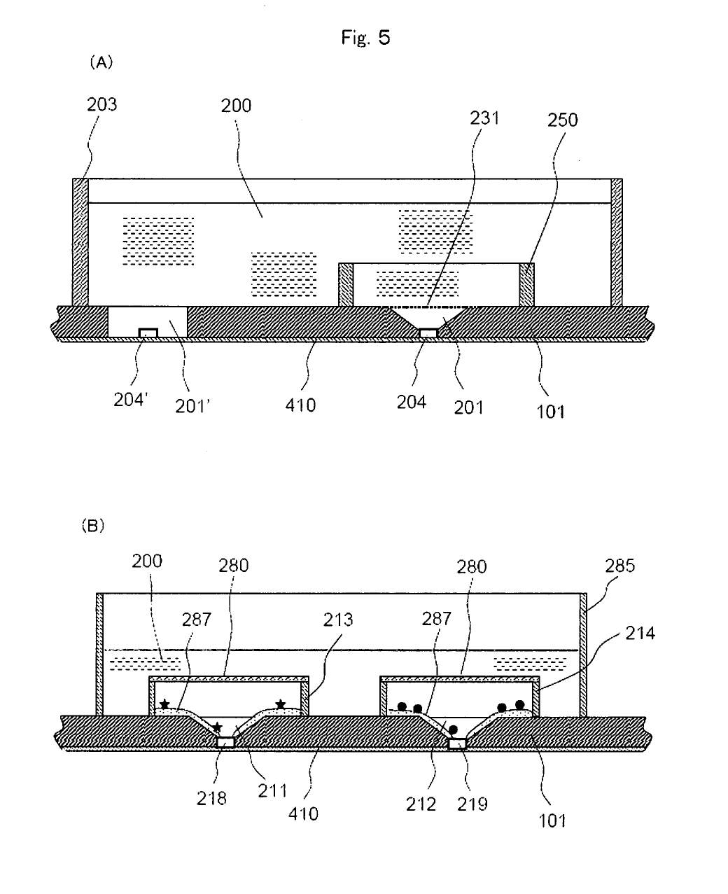

FIG. 4(A) is a cross-sectional view showing a problem caused by increasing the width of a flow channel, and FIG. 4(B) is a cross-sectional view showing an example of means for solving the problem. In FIG. 4(A) and FIG. 4(B), reference numeral 101 represents a substrate, reference numeral 260 represents a groove formed in the substrate 101, and reference numeral 410 represents a laminate film covering the groove 260. The flow channel 205 is formed of the groove 260 and the laminate film 410 covering the groove 260. As shown in FIG. 4(A), in the case where the width of the flow channel 205 is increased, when the laminate film 410 is attached to the chip substrate 101 by thermal pressurization, the laminate film 410 drops into the groove 260 and thus closes the flow channel 205. By contrast, in FIG. 4(B), a beam 400 is provided in a wide portion of the groove 260 and prevents the laminate film 410 from dropping into the groove 260. Thus, the flow channel 205 is not closed.

FIG. 5(A) is a cross-sectional view taken along line (A)-(A) in FIG. 1 and seen in the direction of the arrows thereof. FIG. 5(A) shows the reservoir 203 on the flow channel entrance side, the openings 201 and 201′, and the flow channels 204 and 204′ in more detail. FIG. 5(B) is a cross-sectional view taken along line (B)-(B) in FIG. 1 and seen in the direction of the arrows thereof. FIG. 5(B) shows the culturing tanks 213 and 214, the openings 211 and 212, the flow channels 218 and 219, the semipermeable membrane 280, and the reservoir 285 on the flow channel exit side in more detail.

In the bottom surface of the chip substrate 101, grooves corresponding to the microscopic flow channels 204 and 204′ are formed and are covered with the laminate film 410. Thus, the flow channels 204 and 204′ are formed. The hole 201 for introducing the cell-containing sample buffer solution to the microscopic flow channel 204 is provided at an upstream end of the flow channel 204, and the hole 201′ for introducing the buffer solution with no cells to the microscopic flow channel 204′ is provided at an upstream end of the flow channel 204′. The wall or the reservoir 250 is provided to surround the hole 201 in order to prevent the cell-containing sample buffer solution injected to the hole 201 from being diffused.

In addition to the holes 201 and 201′ and the wall or the reservoir 250 for preventing the cell-containing sample buffer solution injected to the hole 201 from being diffused, the holes 202 and 202′ not shown in FIG. 5(A) are also provided in the reservoir 203. The reservoir 203 is filled with a buffer solution 200 and the buffer solution 200 is supplied to all the holes in the reservoir 203. Therefore, the buffer solution flowing in the microscopic flow channels 204 and 204′ can have a substantially equal flow rate. The buffer solution flowing in the microscopic flow channels 205 and 205′ can also have a substantially equal flow rate. The flow rate of the buffer solution in the microscopic flow channel 204 and that of the buffer solution in the microscopic flow channel 205 can be maintained at a predetermined ratio stably. The hole 201 is cone-shaped to ensure that the sample cells flow to the flow channel 204. The wall 250 may be a low, small reservoir located within the reservoir 203 as shown in FIG. 1, or may be like a simple partitioning plate. A membrane filter 231 covering the hole 201 on the top surface of the chip substrate 101 is provided for removing dust from the sample. The wall 250 prevents the cells from being diffused, and therefore it is not necessary to directly inject the cell-containing sample buffer solution to the hole 201.

The structure of providing a common reservoir at an upstream end of the flow channels is one of the core elements of the cell separation and culturing apparatus according to the present invention. Because the flow channels have a common liquid surface level owing to the common reservoir, the buffer solution can be sent to the plurality of flow channels at the same pressure. This is the simplest liquid delivery system which can be incorporated to the substrate. In order to distinguish the liquids in the flow channels from one another, a partitioning plate lower than the liquid surface level is provided. Owing to this structure, different types of buffer solutions can be flown to the different flow channels at the same pressure. For the buffer solution to be separated by the partitioning plate, a buffer solution having a larger specific gravity than that of the buffer solution forming the common liquid surface level is preferably used. Then, the different types of buffer solutions are not mixed together. The cells basically cause no problem because the cells have a greater specific gravity as they are and precipitate in the container. For chemotactic cells, the partitioning plate is formed to have a height that the cells cannot go beyond. For example, nerve cells cannot go beyond a wall (partitioning plate) having a height of several tens of micrometers. In the case of cells such as E. coli, a sponge-like membrane through which the buffer solution can pass freely but not the cells may be provided over the wall 250. Thus, the cells are prevented from entering different flow channels.

As is clear from FIG. 5(B), the culturing tanks 213 and 214 are covered with the semipermeable membrane 280 at a top surface thereof for protecting the culturing tanks 213 and 214 against contamination during the separation operation. The reservoir 285 is provided to surround the culturing tanks 213 and 214. The reservoir 285 is sufficiently higher than the culturing tanks 213 and 214. The buffer solution put into the reservoir 203 in a sufficient amount on an initial stage of the separation is moved toward the reservoir 285 as the separation proceeds. FIG. 5(B) schematically shows a state where the separation has proceeded to some extent and the culturing tanks 213 and 214 are precipitated in the buffer solution (medium) 200. Reference numeral 287 represents a layer of collagen, polylysine or fibronectin applied on a bottom surface of the culturing tanks 213 and 214. Instead of applying such a substance, the bottom surface of the culturing tanks 213 and 214 may be treated to be hydrophobic. To apply the above-mentioned substances to the bottom surface or to treat the bottom surface to be hydrophobic is convenient to culture cells which form colonies on an agar medium. Hence, in the case where the cells to be separately cultured are bacteria or others which do not form colonies on the agar medium, such application or treatment is not necessary. In FIG. 5(B), the black circles and the stars on the collagen layer 287 applied on the culturing tanks 213 and 214 represent cells separated as described above with reference to FIG. 2.

Next, the gel electrodes will be practically described. A gel electrode section includes holes 206, 206′, 207 and 207′, the microscopic element 208 connecting the holes 206 and 206′, the microscopic element 209 connecting the holes 207 and 207′, and the connection sections 241 and 242 acting as liquid junctions in the cell separation area 222 shown in FIG. 1. On the stage where the chip substrate 101 is produced by injection molding and the cell separation and culturing apparatus 100 is produced by covering the grooves with the laminate film 410, the gel electrode section does not accommodate any gel. In the following example, agarose gel containing an electrolyte is used as an electrolyte solution.

On the negative side of the gel electrode section, i.e., on the side of the microscopic element 209 and the connection section 242, a gel having a composition of 1% agarose, 0.25 M NaCl, and 0.296 M sodium phosphate (pH 6.0) buffer solution is provided. On the positive side, i.e., the side of the microscopic element 208 and the connection section 241, a gel having a composition of 1% agarose, 0.25 M NaCl, and 0.282 M sodium phosphate (pH 8.0) buffer solution is provided. The pH values are made different in order to avoid the phenomenon that bubbles are generated by electrolysis when an electric current flows. Hydrogen ions generated on the positive side are neutralized by the buffer solution having a high pH value before becoming hydrogen molecules. Hydroxy ions generated on the negative side are neutralized by the buffer solution having a low pH value and thus inhibit the generation of oxygen molecules.

The gel electrode is preferably formed of a gel substance containing sugar. In this case, the sugar preferably contains 3% to 50% of nonreducible disaccharide, 1% to 50% of trehalose, 5% to 30% of glycerol, 5% to 40% of ethyleneglycol, or 5% to 30% of dimethylsulfoxide.

Now, it is assumed that gel is injected from the holes 206 and 207 formed in the chip substrate 101 to complete the production of the gel electrode-equipped cell separation and culturing apparatus 100, and then the apparatus 100 is left without being used. The gel is in contact with the air in the openings of the holes 206, 206′, 207 and 207′ and in the flow channels and the connection sections 241 and 242 as liquid junctions in the cell separation area 222. Therefore, the gel starts drying from these areas. In order to store the gel electrode-equipped cell separation and culturing apparatus produced above, the following needs to be done. In order to prevent the gel from drying in the openings of the holes 206, 206′, 207 and 207′, the holes 206, 206′, 207 and 207′ are sealed until immediately before the apparatus 100 is used. In order to prevent the gel from drying in the flow channels and the connection sections 241 and 242 in the cell separation area 222, the apparatus 100 is stored in a sealed container together with a water-containing sheet, such that the gel is not dried. Thus, the apparatus 100 can be easily stored at 4° C. for about 3 months. As the sealed container, a laminate pack is suitable in order to minimize the air space.

In order to prevent the gel from drying and store the apparatus 100 for an extended period of time, gel is supplied with a humectant. As the humectant, for example, about 1% to 10% of disaccharide such as trehalose or sucrose, or oligosaccharide, or about 5% to 10% of glycerin is effective to prevent drying.

For long-time storage, it is preferable to freeze the gel electrode-equipped cell separation and culturing apparatus 100 in a laminate pack. In this case, a problem occurs that ice crystals are generated at the time of freezing and melting and destroy the gel structure. When ice crystals are generated in a gel electrode formed in a tiny area such as in the cell separation and culturing apparatus, the portion in which the ice crystals are generated becomes hollow after the gel electrode is melted. Then, when an electric field is applied to the electrode, the cells enter the hollow portion or such cells unnecessarily flow out to the flow channels of the cell separation and culturing apparatus 100.

In order to prevent this, the gel in the gel electrodes is supplied with a substance for suppressing the crystal growth of ice so as to store the cell separation and culturing apparatus 100 in a frozen state for an extended period of time. This is one of the most important points of the present invention. As the substance for suppressing the crystal growth, substantially the similar substances to those for the humectant are usable. It is most effective to mix a disaccharide such as trehalose or sucrose, or oligosaccharide during the production of gel. Trehalose has a very small function to general animal cells and thus is very effective. The concentration of trehalose may be as low as 1%, and about 50% at the highest. Sucrose is also effective, but is biofunctional to animal cells and may be inappropriate depending on the purpose. By replacing a part of the hydroxyl group of a sugar chain with a sulfuric acid group, the freezing prevention capability can be maintained to reduce the biochemical influence. It is preferable to introduce a sulfuric acid group to the hydroxyl group of these disaccharides. Other sugars such as glycerin and ethyleneglycol are also effective. Dimethylsulfoxide is also effective. It should be considered that ethyleneglycol and dimethylsulfoxide may have a problem of cell toxicity in some cases, but dimethylsulfoxide or the like elutes to a cell sorting flow channel generally in a tiny amount and thus is ignorable.

Practical examples will be described. A cathode electrolyte solution and an anode electrolyte solution having the following compositions are heated and melted in a microwave oven and made into buffer solutions. Separately, the chip substrate 101 is heated on a hot plate heated to 60° C. The cathode electrolyte solution and the anode electrolyte solution in a buffer solution state are respectively injected to the holes 206 and 207 using a syringe and suctioned from the holes 206′ and 207′ to fill the microscopic elements 208 and 209 and the connection sections 241 and 242. Melted gel enters the connection sections 241 and 242 by the capillary phenomenon. After being left at room temperature for 10 minutes, the buffer solutions in the microscopic elements 208 and 209 and the connection sections 241 and 242 are gelated. The flow channel 247 has a larger cross-section than that of the connection 241 and 242, and thus the gelated buffer solution does not go into the flow channel 247.

The improved gel composition will be shown below.

Negative electrolyte solution: 1% trehalose, 0.25 M NaCl, 0.296 M sodium phosphate (pH 6.0), 1% agarose

Positive electrolyte solution: 1% trehalose, 0.25 M NaCl, 0.282 M sodium phosphate (pH 8.0), 1% agarose

The surface of the gel electrode-equipped cell separation and culturing apparatus 100 prepared as above is sealed with an adhesive tape. “Plas-Chamois”, which is a porous plastic towel, is immersed with water, and a 2 cm×2 cm squeezed piece of “Plas-Chamois” is put in a plastic bag having a size of 30 mm×40 mm together with the gel electrode-equipped cell separation and culturing apparatus 100. The plastic bag is sealed with a sealer.

The plastic bag is stored at 4° C. or −20° C. in this state.

The state of the electrode section of the chip is observed with a microscope immediately after the chip is produced but before frozen, and after the chip is stored at 4° C. and −20° C. for 1 month, 3 months, and 6 months. In addition, the reservoir 203 is supplied with a culturing solution to fill the flow channels 204, 205 and 205′, and the hole 201 is supplied with erythrocytes. An application of an electric field to the gel electrodes is turned on and off, so that it is confirmed that the cells are separated to the flow channels 218 and 219. In the chip immediately after being produced, the microscopic elements 208 and 209 and the connection sections 241 and 242 are filled with the gel, with no external damages such as cracks or drying. When an electric field is applied to the gel electrodes, the erythrocytes flowing in the flow channel in the cell separation area 222 are moved to the flow channel 218 and accumulated in the culturing tank 213 via the hole 211. Without the electric field, the erythrocytes are moved to the flow channel 219 and accumulated in the culturing tank 214 via the hole 212. Even in the case where the chip is frozen for six months before use, the cells can be collected in the culturing tank 213 by applying an electric field to the gel electrodes, and in culturing tank 214 by turning off the electric field, similarly to immediately after the production of the chip. In the case where the chip is stored at 4° C., the gel is retracted to the connection sections 241 and 242 in three months according to an observation with a microscope. However, when the cells are caused to flow, the cells can be separated similarly to immediately after the chip is produced.

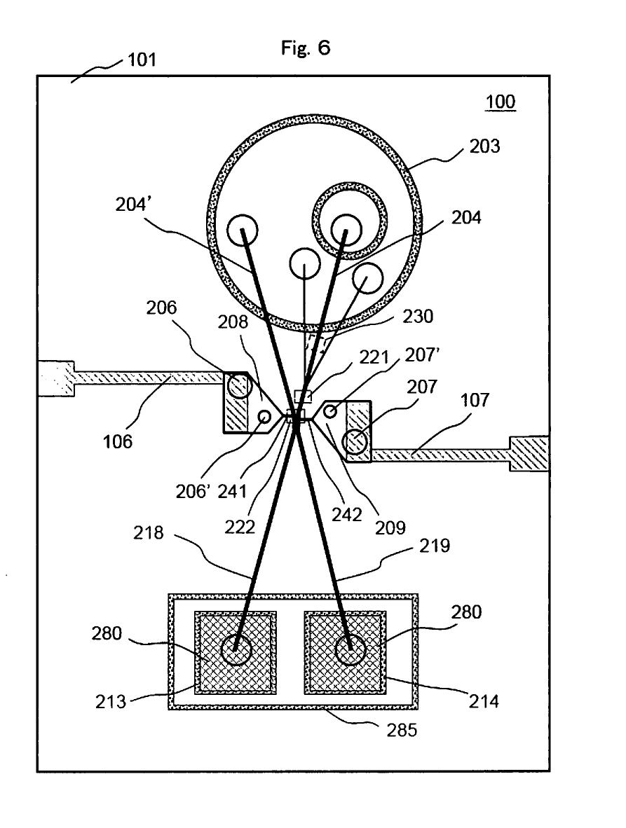

FIG. 6 is a plan view of the cell separation and culturing apparatus 100 in which the gel electrode section has a different structure from that shown in FIG. 1. In this example, the lines 106 and 107 and the electrodes inserted to the holes for introducing the gel in FIG. 1 are formed of a conductive film which is vapor-deposited on the laminate film 410 applied to the bottom surface of the chip substrate 101. Since the laminate film 410 is applied to the bottom surface of the chip substrate 101, the electrodes and the like formed of the conductive film are not actually seen in the plan view of the chip substrate 101. In FIG. 6, however, the electrodes and the like are also shown in order to clarify the relationship thereof with the other elements.

With the structure shown in FIG. 6, pentagonal microscopic elements 208 and 209 are used in place of the microscopic elements 208 and 209 shown in FIG. 1. The pentagonal microscopic elements 208 and 209 are respectively in communication with the openings 206 and 207 formed in the chip substrate 101. The gel is injected through the openings 206 and 207. The openings 206′ and 207′ are respectively in communication with the microscopic elements 208 and 209 for discharging air. The gel injection may be terminated when the openings 206′ and 207′ are overflown with the gel. The connection sections 241 and 242 are projected from the pentagonal microscopic elements 208 and 209, and thus the gel can be in contact with the buffer solution flowing in the flow channel 247. In order to obtain a sufficient electric connection between the gel injected to the pentagonal microscopic elements 208 and 209 and the conductive films 106 and 107 replacing the lines 106 and 107, the conductive films 106 and 107 are vapor-deposited at positions where the gel injected to the pentagonal microscopic elements 208 and 209 are in contact with ends of the conductive films 106 and 107 in an appropriate area. The other ends of the conductive films 106 and 107 act as terminals to be connected to the power supply 215.

Since the laminate film 410 is applied to the chip substrate 101, the terminals at the other ends of the conductive films 106 and 107 are hidden by the chip substrate 101. Although not shown in FIG. 6, an element connected to the terminals for making the terminals connectable with the power supply 215 on the top surface of the chip is provided.

Also with the structure shown in FIG. 6, cells determined to fulfill a predetermined condition in the cell detection area 221 are separated from the other cells in the cell separation area 222, flow down the flow channel 218 and are collected in the culturing tank 213. Cells determined not to fulfill the predetermined condition are collected in the culturing tank 214. The culturing tanks 213 and 214 are covered with the semipermeable membrane 280 at a top surface thereof for protecting the culturing tanks 213 and 214 from contaminants during the cell separation. During the cell separation operation, the semipermeable membrane 280 is provided for protecting the culturing tanks 213 and 214 from contaminants. During the cell culturing operation performed in a culturing device after the flow channels 218 and 219 communicating with the culturing tanks 213 and 214 are closed and the culturing tanks 213 and 214 are cut off from the separation and culturing apparatus 100, the semipermeable membrane 280 acts as a membrane for supplying the cells with a medium as described later. The reservoir 285 is provided to surround the culturing tanks 213 and 214. When the cells not fulfilling the predetermined condition do not need to be cultured, the culturing tank 214 may be omitted. Instead of providing two routes, i.e., a route for cells selected as fulfilling the predetermined condition and a route for non-selected cells as described above with reference to FIG. 1 and FIG. 2, there may be three or more routes.

Using the cell separation and culturing apparatus 100 in FIG. 1, an algorithm for cell recognition and separation which is based on image recognition will be described. As described above with reference to FIG. 1, a cell suspension is injected to the hole 201. The wall 250 is provided to surround the hole 201 in order to prevent the cell suspension from being diffused. The wall 250 is provided within the reservoir 203, and the liquid surface level in the hole 201 is equal to the liquid surface level in the reservoir 203. The cells flow from the hole 201 to the flow channel 204 and are merged with the buffer solution in the flow channel 205, which forms a side flow, before the cell detection area 221. Thus, the cells are pushed to the center of the flow channel (FIG. 3).

The cells passing through the cell detection area 221 from the flow channel 204 are imaged by a CCD camera. A CCD camera capable of capturing images at, for example, 200 frames per second is used. With such an imaging capability, each of the cells can be recognized even when the flow rate of the buffer solution passing through the cell detection area 221 is about 1 mm/sec.

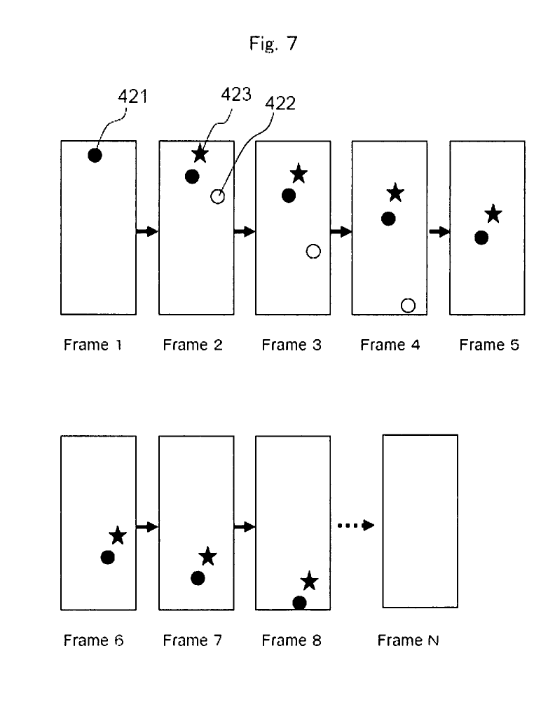

FIG. 7 shows an algorithm for recognizing cells from an image captured by the CCD camera and numbering and identifying each of the cells. In FIG. 7, the images captured one after another are represented as “frame 1”, “frame 2”, . . . , “frame N”. In each frame, cells are displayed. In frame 1, only one cell represented with a black circle is displayed. This cell represented with the black circle is recognized as an image and numbered as No. 421. In frame 2, a cell represented with a white circle and a cell represented with a star are displayed in addition to cell 421. Frame 2 is the first frame in which the cell represented with the white circle and the cell represented with the star appear, and these cells appear simultaneously. These cells are recognized as images and numbered. The cell which is seen downstream with respect to the other, i.e., which is detected first, is assigned with a smaller number. Here, the cell represented with the white circle appears downstream with respect to the cell represented with the star. Thus, the cell represented with the white circle is numbered as No. 422, and the cell represented with the star is numbered as No. 423. In frame 3, no new cell is recognized. It is understood by comparing frame 2 and frame 3 that cell 422 is moved faster than cell 421 and cell 423. In frame 4, no new cell is recognized. Cell 422 is moved fast and thus is barely seen in frame 4. Cell 421 and cell 423 are seen as moving almost at the same speed. The cells can be recognized in up to frame 8. The image recognition is performed using, as indices, the luminance center of gravity, area size, circumferential length, longer diameter, and shorter diameter.

Based on the moving velocity of each cell recognized as an image and numbered, the time necessary for the respective cell to reach the cell separation area 222 (more strictly, the connection section 241 or 242) is found. The cells are divided into the cells sent to the recovery hole 211 and the cells sent to the recovery cell 212 by applying a negative electric field or no electric field to the gel electrode in the connection 241 and applying a positive electric field or no electric field to the gel electrode in the connection 242. In other words, the moving velocity (V) of each of the cells numbered based on the images captured at an interval of a predetermined time period is calculated, and the cells are separated by applying a voltage at a timing of (L/V) to (L/V+T). The length (L) and the application time (T) are input in advance in relation to the cell moving velocity (V).

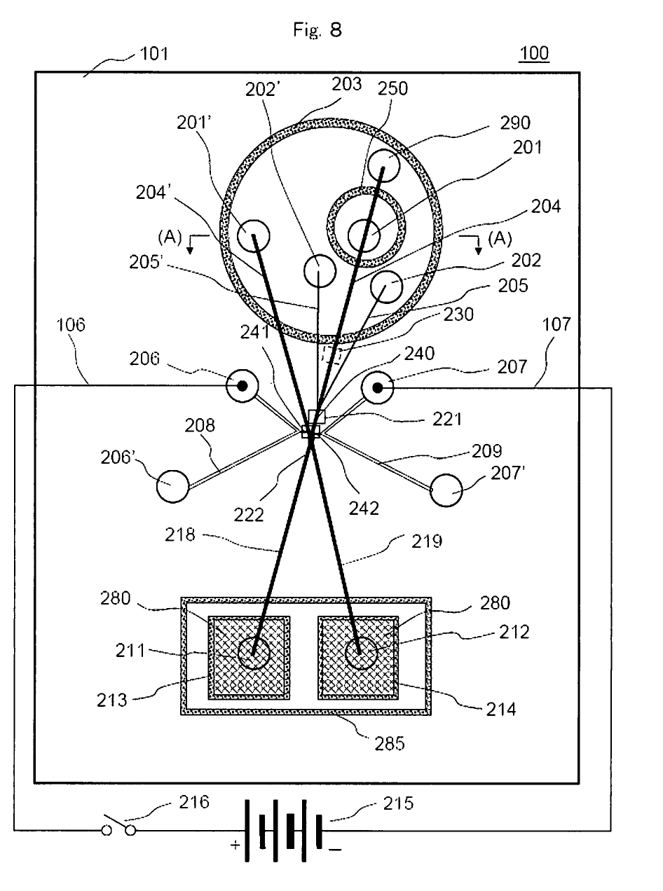

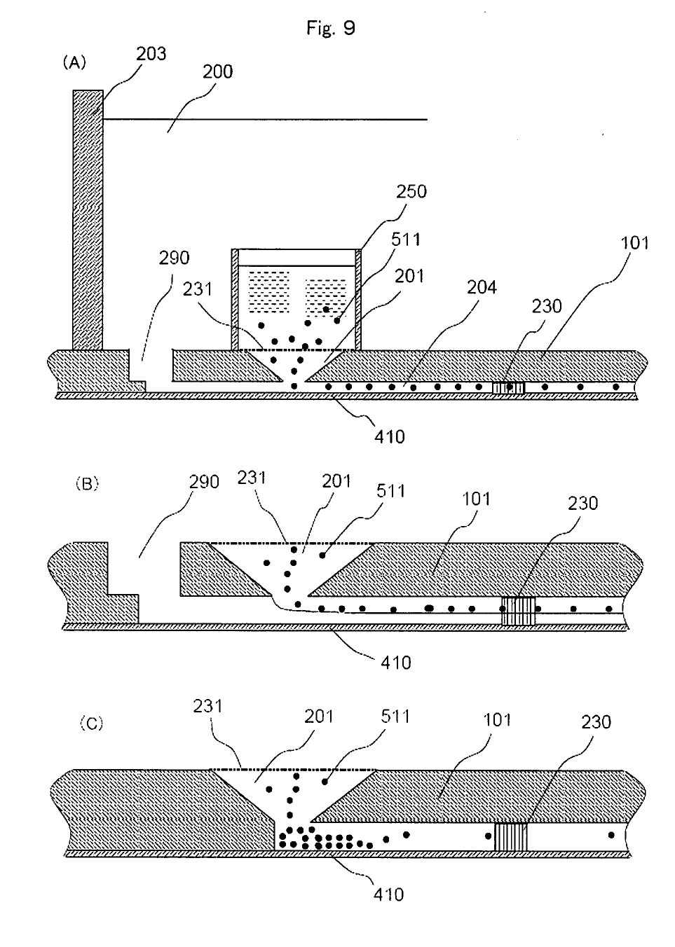

FIG. 8 is a plan view schematically showing one example of a system structure of a cell separation and culturing apparatus according to the present invention. As compared to the apparatus in FIG. 1, the apparatus in FIG, 8 has a special arrangement for the introduction of sample cells. FIG. 9(A), FIG. 9(B) and FIG. 9(C) are partial cross-sectional views illustrating the arrangement of the sample cell introduction section. The elements bearing the identical reference numerals as those in FIG. 1 are identical elements thereto or act in an identical manner thereto.

As is clear from a comparison between FIG. 8 and FIG. 1, in the cell separation and culturing apparatus shown in FIG. 8, the flow channel 204 is extended to a further upstream position and an opening 290 is provided for introducing a buffer solution. Except for this, the apparatus in FIG. 8 is the same as the apparatus in FIG. 1. As understood from FIG. 9(A), the flow channel 204 is in communication with the reservoir 250 via the opening 201, and is further extended to be in communication with the opening 290. In FIG. 1 and FIG. 2, there are two routes, i.e., a route for cells selected as fulfilling the predetermined condition and a route for non-selected cells. Alternatively, there may be three or more routes.

FIG. 9(B) schematically shows the buffer solution and the cells flowing from the openings 201 and 290 to the flow channel 204. As understood from this, in this embodiment, the layer of the buffer solution flowing from the opening 290 to the flow channel 204 prevents the buffer solution and the cells flowing from the opening 201 to the flow channel 204 from contacting the laminate film 410. Namely, the buffer solution and the cells flowing from the opening 201 to the flow channel 204 flow on the layer of the buffer solution flowing from the opening 290 to the flow channel 204. Owing to this arrangement, the cells are prevented from contacting the laminate film 410, and the resultant jamming of cells is avoided.

FIG. 9(C) schematically shows that the cells flowing from the opening 201 to the flow channel 204 contacts the laminate film 410 and thus are jammed. Once one of the cells contacts the laminate film 410 and stays there, the other cells are caught by the first cell and stay there one after another. Finally, the flow of the cells is stopped.

The structure in FIG. 8 is described as being realized by extending the flow channel 204 in FIG. 1 to an further upstream position. Clearly, the flow channel 204 in FIG. 6 may be similarly extended. The point is that the buffer solution is introduced upstream with respect to the position of cell introduction, so that a flow layer of the buffer solution is formed before the cells are introduced. Thus, the cells are prevented from contacting the bottom surface of the flow channel.

(Example of Cell Modification)

In the following, cells are modified with a fluorescent dye, gold microparticles or non-gold nanoparticles, and aptamer is used to detect the cells with fluorescence or scattered light. In order to identify or separate cells, some distinguishing index is needed. In the following example, a substance decomposable under a mild condition is used for labeling a surface antigen, and the labeling substance for the surface antigen is decomposed and thus removed under a physiological condition with no influence on the cells. Practically, polynucleotide capable of forming various steric structures is used as the labeling substance. Here, polynucleotide is used as an element generally conceived as an aptamer. For example, various types of synthetic polynucleotides as follows are prepared: the total length is 80 bases; 20 bases on the 3′ terminus side and 20 bases on the 5′ terminus side are of a regulated known basic sequence; and 40 bases at the center are of a random sequence. These synthetic polynucleotides are passed through a column, on an inner surface of which a surface antigen of the cell to be separated is immobilized. As a result, a polynucleotide having a sequence with affinity to the surface antigen of the cell to be separated is captured on the inner surface of the column. This column is alkali-treated to separate and thus recover the captured polynucleotide. The recovered polynucleotide is PCR-amplified. Thus, a polynucleotide specifically bound to the cell surface antigen is obtained. Namely, an aptamer as a surface antigen labeling substance which is decomposable under a mild condition is obtained.

In order to obtain an aptamer (polynucleotide) having a higher specificity and binding strength, an evolutionary engineering means of intentionally lowering the fidelity at the time of the PCR amplification to change the sequence and repeating the affinity purification may be additionally used. In some cases, the binding strength may be increased by modifying and thus charging a base portion bound to the surface antigen. Alternatively, the binding strength may be increased by using nucleotide in which the sugar chain portion of the bases is modified.

The backbone structure of the obtained structure-recognizable polynucleotide may be of a ribonucleotide type or a deoxyribonucleotide type. In general, the ribonucleotide type is more advantageous as being usable for various structures, but may occasionally be difficult to use because RNase in the periphery thereof causes unpredictable decomposition. The deoxyribonucleotide type is more easily usable because there are not many DNase outside the cells and deactivation is easily done.

The structure-recognizable polynucleotide (aptamer) obtained in this manner as the labeling substance is modified with a fluorescent substance, or gold or magnetic nanoparticles as an identifying substance, thus to produce an identifying element. The identifying element is mixed with the sample cells to identify the cells having a site bound to the labeling substance and to separate such cells by the cell separation and culturing apparatus based on the identification information.

After the separation, the cells are treated with nuclease to decompose and thus remove the labeling polynucleotide bound to the surface antigen. In the case where the labeling polynucleotide is of the ribonucleotide type, RNase is used for decomposition. In the case where the labeling polynucleotide is of the deoxyribonucleotide type, DNase is used for decomposition. When a modified nucleotide is used in order to increase the stability, it is important that the modified nucleotide should not entirely inhibit the decomposition by the nuclease. The nucleotide structure which has a possibility of inhibiting the effect of the nuclease should be introduced to only a part of the aptamer molecule, if introduced. With such an arrangement, the aptamer molecule is decomposed to be of a sufficiently low molecular weight when considered as a whole, although nuclease may not work for a part of the aptamer.

By this method, the structure-recognizable polynucleotide (aptamer) as the labeling substance for the cell surface antigen can be easily removed with nuclease. Since RNase and DNase cannot pass through the cell membrane, the RNAs or DNAs in the cell are not damaged. Since the RNAs or DNAs are not considered to be exposed to the cell surface, it is considered that the cell itself is not influenced by the nuclease due to the structure-recognizable polynucleotide (aptamer) bound to the cell surface antigen. Therefore, the cells are prevented from being denatured due to the treatment performed to separate the cells.

Preparation of an aptamer for the cell surface antigen CD4 will be described. This aptamer is one of aptamers useful as a labeling substance.

As the aptamer as a labeling substance, the aptamer for the cell surface antigen CD4 described in “Staining of cell surface human CD4 with 3′-F-pyrimidine-containing RNA aptamers for flow cytometry”, Nucleic Acids Research 26, 3915-3924 (1998) is used. This aptamer is of a ribonucleotide, i.e., is an RNA aptamer. In the above-mentioned article, the aptamer is made identifiable with fluorescence by introducing GDP-β-S as an identifying substance to the 5′ terminus of the RNA aptamer by in vitro transcription. Namely, at this point, a thiophosphoric acid group is inserted to the 5′ terminus of the RNA aptamer. The thiophosphoric acid group is reacted with biotin, to which an iodoacetyl group is introduced, and thus a 5′ biotinated RNA aptamer is obtained.

A conjugate of phycobiliprotein and streptoadipine as a fluorescent colorant is reacted with the above-obtained aptamer, and a phycobiliprotein-modified RNA aptamer is obtained through a biotin-adipine reaction. Among phycobiliproteins, β-phycoerythrin is a fluorescent protein type fluorescent substance having a high absorbance of 2.41×10⁶M⁻¹ cm⁻¹ and a high quantum efficiency of 0.98 and thus is suitable for high sensitivity detection, but has problems of a molecular weight which is as high as 240 K Dalton, and the non-specific adsorption and instability because of being protein. Here again, a phycobiliprotein-modified RNA aptamer is usable as a practical example, but this is equivalent to using particles of about 10 nm as an identifying substance in terms of size because the phycobiliprotein-modified RNA aptamer has a molecular weight of as great as 240 K Dalton. Therefore, in addition to phycobiliprotein, fluorescent colorant-containing particles having a diameter of 10 nm, gold nanoparticles having a diameter of 10 nm, and magnetic particles having a diameter of 10 nm are also used as an identifying substance.

In this example, an identifying element using phycobiliprotein or nanoparticles as an identifying substance will be described.

(i) Phycobiliprotein-modified RNA aptamer: The method described in the above-mentioned article may be used, but another method is used in this example. A synthetic RNA aptamer can be obtained with certainty by chemical synthesis. An amino group is introduced to the 5′ terminus of the synthetic RNA aptamer at the time of chemical synthesis thereof. The amino group introduced to the 5′ terminus is reacted with a bivalent reagent such as N-(8-maleimidocapryloxy)sulfosuccinimide, and a maleimido group reactable with an SH group is introduced to the 5′ terminus of the RNA aptamer. Separately, β-phycoerythrin having an SH group introduced thereto is prepared. For introducing the SH group, the amino group of the β-phycoerythrin is modified with 2-iminothiorane. The RNA aptamer having the maleimido group introduced thereto, and the β-phycoerythrin having the SH introduced thereto by modification with 2-iminothiorane, are mixed together at pH 7, and thus a β-phycoerythrin-modified RNA aptamer is obtained.

(ii) Gold nanoparticle-modified RNA aptamer: A method for preparing gold nanoparticle-modified RNA aptamer referring to the method described in Tonya M. Herne and Michael J. Tarlov, J. Am. Chem. Soc. 1997, 119, 8916-8920 and the method described in James J. Storhoff, J. Am. Chem. Soc. 1998, 120, 1959-1964 will be described. To a gold nanoparticle (20 nmφ) suspension, a synthetic RNA aptamer having an SH group at the 5′ terminus and 6-mercapto-1-hexanol are added, and left for 1 hour. The molar ratio of the synthetic RNA aptamer and 6-mercapto-1-hexanol is 1:100. In the case where the gold nanoparticles coagulate or in the case where the synthetic RNA aptamer and the gold nanoparticles are not bound together, the molar ratio may be optionally varied to find an optimum condition. Since the gold nanoparticles easily coagulate, the synthetic RNA aptamer is added while stirring the suspension, such that the concentration gradient of the potassium carbonate buffer solution or the concentration gradient of the synthetic RNA aptamer is not generated. The reaction is caused at the molar ratio of the gold nanoparticles and the synthetic RNA aptamer of 1:100. Namely, the reaction occurs where the number of the gold nanoparticles and the number of the synthetic RNA aptamer molecules are at the ratio of 1:1000. The synthetic RNA aptamer having an SH group is obtained by chemical synthesis. After the reaction, the resultant substance is centrifuged at 8000 G for 1 hour to remove the supernatant. The resultant substance is suspended again in a 10 mM potassium carbonate buffer solution (pH 9) containing 0.1 M NaCl, centrifuged again to remove the supernatant, and suspended in a 10 mM potassium phosphate buffer solution (pH 7.4) containing 0.1 M NaCl. The resultant substance is used as a stock.

(iii) Non-gold nanoparticle-modified RNA aptamer: For example, nanoparticles such as quantum dots are generally formed of inorganic nanoparticles. A product covered with polyethyleneglycol having biotin introduced thereto is already commercially available under the trade name of, for example, EviFluor from Evident Technologies. Nanoparticles with biotin may be used with a streptoadipine-bound RNA aptamer. A method for preparing an RNA aptamer bound to streptoadipine will be described. The RNA aptamer having a maleimido group introduced to the 5′ terminus, and streptoadipine having an SH introduced thereto by modification with 2-iminothiorane, are mixed together at pH 7 by the method described in (i) above, and thus a streptoadipine-bound RNA aptamer is obtained. The streptoadipine-bound RNA aptamer and the nanoparticles with biotin are mixed together, and thus a nanoparticle-labeled RNA aptamer is obtained as an identifying element.

When nanoparticles having a carboxylic group introduced thereto is used, a nanoparticle-labeled RNA aptamer as an identifying element can be obtained by a well known method of active-esterifying the carboxylic group with carbodiimide and reacting the active ester with 5′ aminized RNA aptamer.

So far, methods for preparing nanoparticle-modified RNA aptamers have been described. Also in the case of a DNA aptamer formed of deoxyribonucleotide, an SH group or an amino group can be introduced to the 5′ terminus when synthesizing a DNA aptamer with a synthesizing apparatus, like in the case of the above-described RNA aptamers. Therefore, with deoxyribonucleotide, a phycobiliprotein-modified DNA aptamer, a gold nanoparticle-modified DNA aptamer and a non-gold nanoparticle-modified DNA aptamer can be prepared in a similar manner.

An RNA aptamer may be produced by an established method other than the above-described synthesis methods. According to the established method, a single chain DNA having T7 promoter at the 5′ terminus is synthesized, and then is transcribed to an RNA using RNA polymerase.

Now, an identifying element using an RNA aptamer as a labeling substance for labeling the cell surface antigen CD4 and using β-phycoerythrin as an identifying substance for separating and recovering cells having the RNA aptamer bound thereto will be described. The cell surface antigen CD4-presenting cells are specifically labeled with the above-mentioned β-phycoerythrin-modified RNA aptamer, and separated using a cell separation and culturing apparatus including the plastic chip substrate 101 as shown in FIG. 1, FIG. 6 or FIG. 8.

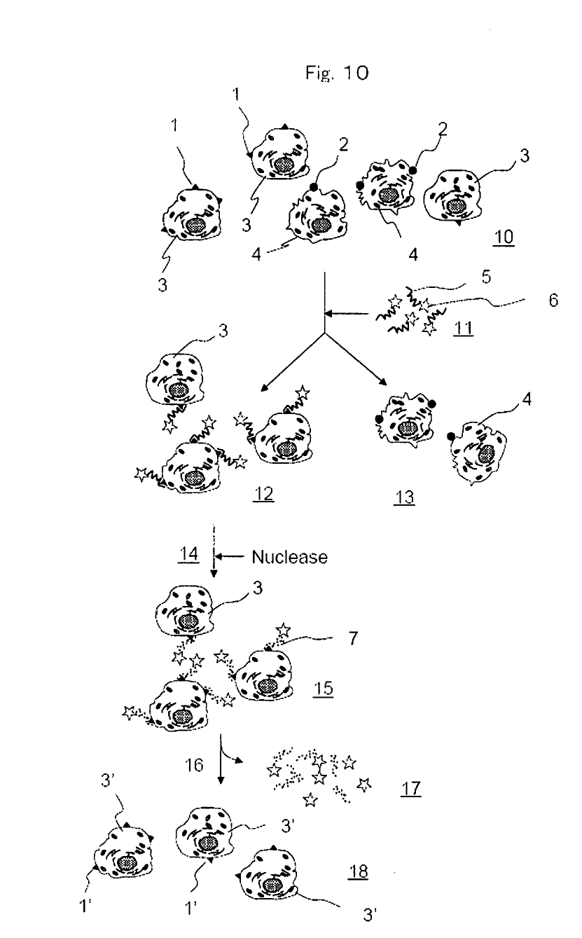

FIG. 10 illustrates a flow of processing for specifically labeling the cell surface antigen CD4-presenting cells with a β-phycoerythrin-modified RNA aptamer and separating the cells by a cell separation and culturing apparatus. A top part of FIG. 10 shows two types of cells 3 and 4 mixed in a sample 10. The cells 3 each have a cell surface antigen CD4 represented with black triangles and reference numeral 1. The cells 4 each have a non-CD4 cell surface antigen 2 represented with black circles. This sample 10 is mixed with a β-phycoerythrin-modified RNA aptamer 11 as described above. The RNA aptamer is represented with reference numeral 5, and β-phycoerythrin is represented with reference numeral 6. The concentration of the labeling substance 11 is 100 nM. A reverse-Y-shaped double headed arrow is shown below the top part of FIG. 1, and a leftward arrow is directed to the common part of the reverse-Y-shaped double headed arrow. The leftward arrow indicates that the β-phycoerythrin-modified RNA aptamer 11 is mixed with the sample 10.

As a result, to the CD4 antigen 1 existing on the surface of the cells 3, the β-phycoerythrin-modified RNA aptamer as the labeling substance is bound. The labeling substance RNA aptamer is not bound to the surface antigen 2 other than CD4. The β-phycoerythrin as an identifying substance for modifying the labeling substance RNA aptamer, when excited by second harmonic of 532 nm from a YAG laser, emits strong fluorescence having a wavelength close to 575 nm. Utilizing this, the cell separation and culturing chip can separate the CD4-presenting cells from the other cells by detecting the fluorescence. Below the reverse-Y-shaped double headed arrow, reference numeral 12 represents a group of cells 3 bound to the labeling substance RNA aptamer, and reference numeral 13 represents a group of cells 4 not bound to the labeling substance RNA aptamer.

Next, the CD4-presenting cells collected in the culturing tank 213 of the cell separation and culturing apparatus 100 are cut off from the cell separation and culturing apparatus 100 while being contained in the culturing tank 213 and put into an arbitrary culturing device. Immediately after this, nuclease 14 is put into the culturing device and introduced to the culturing tank 213 via the semipermeable membrane 280, so that the nuclease 14 acts on the CD4-presenting cells. The RNA aptamer has a steric structure and therefore in some cases is not sufficiently decomposed only with such a type of nuclease as ribonuclease A for decomposing a single chain RNA. Therefore, it is effective to use an enzyme for decomposing both a single chain RNA and a double chain RNA. In this example, an enzyme having the trade name Benzonase (registered trademark; European Patent No. 0229866, U.S. Pat. No. 5,173,418) obtained by mass-producing the nuclease derived from Serratia marcescens described in The Journal of Biological Chemistry 244, 5219-5225 (1969) in a genetic engineering manner is used. This enzyme acts at 37° C. and is usable in a neutral area of pH 6 to 9, and thus is easily usable for cells. The enzymatic activity is lost by highly concentrated phosphoric acid or monovalent metal ions. Therefore, in this example, a non-phosphoric acid-system buffer solution, for example, 10 mM HEPES (pH 7.4) containing 0.15 M NaCl, 2 mM MgCl₂ and 1 mg/ml BSA is used. When it is unavoidable to use a phosphoric acid-system buffer solution, such a buffer solution is used under the conditions that the concentration of potassium phosphate/sodium is limited to 5 mM and that 0.15 M NaCl, 2 mM MgCl₂ and 1 mg/1 ml BSA are contained. Benzonase (registered trademark) is used in an amount of 10 to 100 u/ml. Alternatively, a mixture of ribonuclease A and ribonuclease T1 is usable, but nuclease derived from Serratia marcescens is more generally usable.

Optionally, serum is usable instead of a buffer solution. In this case, nuclease inhibitor in the serum may have an influence. Therefore, it may be necessary to adjust the amount of Benzonase (registered trademark) nuclease for each lot of serum. Generally when serum is used, a good result is obtained with an amount of Benzonase of 100 to 400 u/ml.

In FIG. 10, nuclease is shown with a downward arrow below the group 12 of cells 3 bound to the labeling substance RNA aptamer. This arrow indicates the treatment of adding nuclease, and this treatment is represented with reference numeral 14. Owing to the action of nuclease, the labeling substance RNA aptamer 11 bound to the CD4 antigen 1 on the surface of the cells 3 is decomposed. In FIG. 10, the decomposed RNA aptamer is shown as a collection of dots and represented with reference numeral 7. Reference numeral 15 represents a mixture of the cells 3, the decomposed labeling substance RNA aptamer 7, and the identifying substance β-phycoerythrin 6.

The aptamer introduced to the culturing tank 213 via the semipermeable membrane 280 and decomposed in the culturing tank 213 by the action of nuclease is then discharged via the semipermeable membrane 280. The culturing device accommodating the culturing tank 213 is preferably of a shaking type in order to promote the introduction of the nuclease to the culturing tank 213 via the semipermeable membrane 280, the decomposition of the aptamer in the culturing tank 213, and the discharge of the decomposed aptamer and the identifying substance β-phycoerythrin from the culturing tank 213. Reference numeral 18 represents a collection of the cells 3 remaining in the culturing tank 213 and recovered as still having the CD4 antigen 1 on the surface thereof. Here, the cells are represented with 3′ and the CD4 antigen is represented with 1′ in order to indicate that the cells and the CD4 antigen are not exactly the same before and after the action of nuclease, because the cells and the CD4 antigen may possibly be influenced by the nuclease even though slightly.

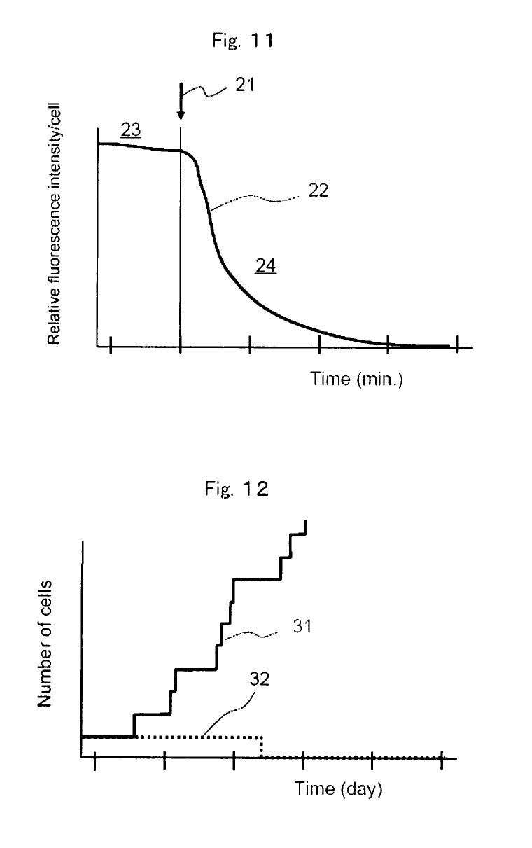

FIG. 11 shows an examination result of the time-wise change of the fluorescence intensity of the identifying substance β-phycoerythrin bound to the cell surface, the change being caused by the addition of nuclease. In this example, a cell is put on a plate and the cell surface is observed with a fluorescent microscope. The accumulated value of the fluorescence intensity obtained from the entire cell is found. When the aptamer portion is decomposed by nuclease, the identifying substance β-phycoerythrin is diffused from the cell surface and becomes undetectable. Utilizing this phenomenon, how nuclease decomposes the aptamer portion can be observed by tracing the fluorescence intensity at the cell surface. In FIG. 11, the horizontal axis represents the time, and the vertical axis represents the fluorescence intensity per cell, which is the accumulated fluorescence intensity from one cell. Namely, using the cell surface antigen CD4-presenting cells to which the β-phycoerythrin-modified RNA aptamer decomposed by the cell separation and culturing apparatus is bound (the group of cells 3 to which the labeling substance RNA aptamer 12 is bound in FIG. 10), the fluorescence intensity at the cell surface is traced in accordance with the time by the fluorescent microscope (exciting wavelength: 532 nm; fluorescence wavelength: 575 nm; a bandpass filter is used). In order to avoid discoloration by the fluorescence, the time of radiation of the exciting light is minimized. For example, the fluorescence intensity is measured while radiating light for 1 second at an interval of 1 minute.

Curve 22 represents the time-wise change of the fluorescence intensity. Arrow 21 represents the timing at which Benzonase (registered trademark) is added. Even if the time of radiation of the exciting light having a wavelength of 532 nm is short, it is difficult to completely avoid discoloration. Even without using Benzonase (registered trademark) (time zone 23), the fluorescence intensity is slightly decreased as the time passes. When Benzonase (registered trademark) nuclease is added at time 21, the fluorescence intensity detectable from the cell is rapidly decreased as shown in time zone 24, although being slightly delayed.

This result indicates the following: on the stage of separating the cell surface antigen CD4-presenting cells to which the β-phycoerythrin-modified RNA aptamer is bound using the cell separation and culturing apparatus, there is no problem with the function of β-phycoerythrin as the identifying substance; when nuclease is added, the RNA aptamer portion (reference numeral 5 in FIG. 10) of the β-phycoerythrin-modified RNA aptamer bound to the cell surface is decomposed and the β-phycoerythrin 6, which is a fluorescent substance, is diffused to the solution.

FIG. 12 shows that the cell surface antigen CD4-presenting cells obtained by removing the β-phycoerythrin-modified RNA aptamer are culturable in the culturing tank 213. The horizontal axis represents the time, and the vertical axis represents the number of cells. Based on the characteristic shown in FIG. 11, the time at which the β-phycoerythrin-modified RNA aptamer is considered to be removed to a sufficient level as a result of the addition of Benzonase (registered trademark) nuclease is evaluated in advance.

In this manner, the labeling substance RNA aptamer is bound to the cells to recognize the surface antigen, and the aptamer is decomposed and removed with ribonuclease when cell labeling becomes unnecessary. Thus, the cells can be returned to a pre-separation natural state in which the cells can be divided. In addition, according to the present invention, the cells obtained by the cell separation performed using the cell separation and culturing apparatus are cultured while being accommodated in the culturing tank used for collecting the cells. Therefore, the cells can be prevented from being contaminated, with certainty.

In the following example, the aptamer as the labeling substance is of an RNA type binding to EpCAM, and the identifying substance is magnetic particles (diameter: about 100 nm). The purpose is to separate and detect cancer-derived cells circulating in blood and having EpCAM as a surface antigen.

An RNA aptamer bound to EpCAM is prepared as follows. A 26-base sequence (SEQ. ID. NO:1) containing a T7 promoter sequence is introduced to the 5′ terminus of a single chain DNA having a random sequence of 40 bases, and a 24-base PCR priming site (SEQ. ID. NO:2) is introduced to the 3′ terminus of the single chain DNA. As a result, a sequence of 90 bases in total is synthesized. The sequence to be introduced to the 5′ terminus of the random sequence of 40 bases is shown as SEQ. ID. NO:1. TAATACGACT CACTATAGGG AGACAA (SEQ. ID. NO:1)

The sequence to be introduced to the 3′ terminus of the random sequence of 40 bases is shown as SEQ. ID. NO:2. NTTCGACAGG AGGCTCACAA CAGG (SEQ. ID. NO:2)

The obtained 90-base sequence is transcribed to an RNA with RNA polymerase using the T7 sequence. For the transcription to the RNA, 100 μl of T7 polymerase is caused to act on 100 μmol of DNA at a scale of 500 μl. As the substrate, 3 mM of each of 2′-F-CTP and 2′-F-UTP and 1 mM of each of ATP and GTP are used. The transcription is performed at 25° C. for 10 hours. After the transcription to the RNA, the DNA is decomposed with DNaseI, and the RNA transcription product is recovered with electrophoresis. The recovered RNA transcription product is thermally denatured, and then passed through an EpCAM immobilized sepharose CL4B column in PBS (pH 7.4) containing 2 mM of MgCl₂. A bound transcribed RNA component is eluted with a solution containing 7 M urea. The obtained transcribed RNA component is reserve-transcribed, and PCR-amplified with a primer pair having a complementary sequence to the known sequence portions at both ends. The obtained PCR product is again transcribed with the T7 promoter, and captured with an EpCAM immobilized sepharose CL4B column in a similar manner. Then, the bound transcribed RNA component is recovered. The steps of transcription—capturing—recovery—PCR amplification are repeated 15 times, and thus an RNA aptamer specifically reactive with EpCAM is obtained.

To the 5′ terminus of the obtained aptamer, a thiophosphoric acid group is inserted with in vitro transcription described in “Staining of cell surface human CD4 with 3′-F-pyrimidine-containing RNA aptamers for flow cytometry”, Nucleic Acids Research 26, 3915-3924 (1998). The thiophosphoric acid group is reacted with biotin having an iodoacetyl group introduced thereto, and thus a 5′ biotinated RNA aptamer is obtained. The 5′ biotinated RNA aptamer is reacted with streptoadipine conjugate magnetic beads, and thus an RNA aptamer which has magnetic particles as the identifying substance and is specifically reactive with EpCAM is obtained.

The reaction of RNA aptamer-labeled magnetic particles with EpCAM-positive cancer cells will be described. 10 ml of blood is suspended in 5 times the volume of culturing solution, and RNA aptamer-labeled magnetic particles corresponding to EpCAM are added and stirred mildly for 30 minutes. The resultant suspension is put to a tube having an inner diameter of 2 mm, and the magnetic particles in the tube are captured by neodymium-system magnet arrays located along the tube at an interval of 1 cm. The cells to which the recovered magnetic particles are bound are separated from the magnetic particles as shown in FIG. 10, washed with the culturing solution, and the cells are separated by the cell separation and culturing apparatus 100 in FIG. 1. Whether each cell is to be separated or not is determined by the shape recognition based on an image. As described above, the cells separated and collected in the culturing tank 213 by the cell separation and culturing apparatus 100 are cut off from the cell separation and culturing apparatus 100 while being contained in the culturing tank 213 and put into an arbitrary culturing device. Immediately after this, Benzonase (registered trademark) nuclease is added to the culturing device to decompose the aptamer, and thus biological cells are obtained. The aptamer introduced to the culturing tank 213 via the semipermeable membrane 280 and decomposed in the culturing tank 213 by the action of nuclease is discharged via the semipermeable membrane 280. The culturing device accommodating the culturing tank 213 is preferably of a shaking type in order to promote the introduction of the nuclease to the culturing tank 213 via the semipermeable membrane 280, the decomposition of the aptamer in the culturing tank 213, and the discharge of the decomposed aptamer and the identifying substance β-phycoerythrin from the culturing tank 213. Cancer cells are durable against long-time culturing, and some cells start to be divided soon in the culturing tank 213.

In general, biological cells circulating in the blood are mostly derived from cancer cells, except for hemopoietic cells. In the blood, cells are not peeled off while being alive from the endothelial surface of the blood vessel; and even if peeled off, the cells are decomposed in the blood owing to the protection mechanism. By contrast, cancer cells are peeled off while being alive, exhibit resistance even in the blood, and circulate in the blood vessel while being alive. However, the cancer cells are existent in a small quantity and are not suitable for biopsy. If the cancer-derived cells circulating in the blood can be concentrated and cultured for a certain time period, it can be found whether a lesion exists somewhere in the body although the site of cancer cannot be not specified.

In the case where the identifying substance of the identifying element is particles or magnetic particles, an image of the particles, scattered light detection, or magnetic detection of the identifying substance for the identifying element is usable to identify the cells to which the labeling substance for the identifying element is bound.

(Example of Cell Culturing Device)

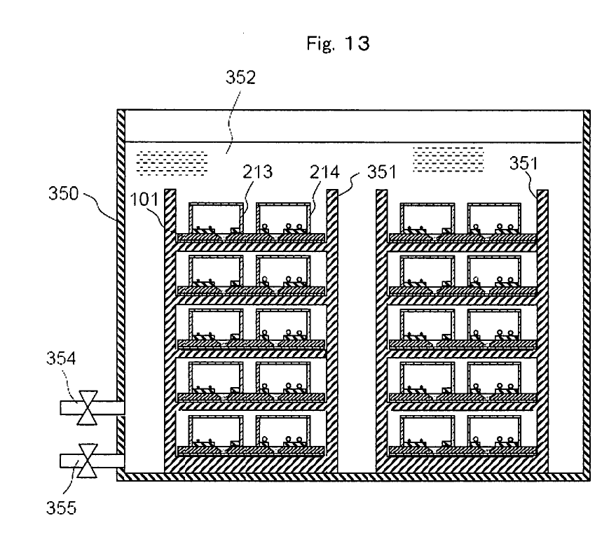

FIG. 13 schematically shows an example of cell culturing device. Reference numeral 350 represents the culturing device. FIG. 13 shows the culturing tanks 213 and 214 together with the chip substrate 101 after being cut off from the cell separation and culturing apparatus and put into the culturing device 350. The culturing tanks 213 are put on a rack 351 and placed in the culturing device 350. In FIG. 13, each rack 351 has five stages of tables. The culturing device 350 includes an air supply pipe 354 for supplying air containing 5% of CO₂ and a supply pipe 355 for supplying a medium 352. Each pipe has an open/close valve. The rack 351 is not absolutely necessary to culture the cells separated and put into the culturing tanks 213 and 214 which are accommodated in the culturing device 350, and the culturing tanks 213 and 214 may be appropriately located in the culturing device 350.

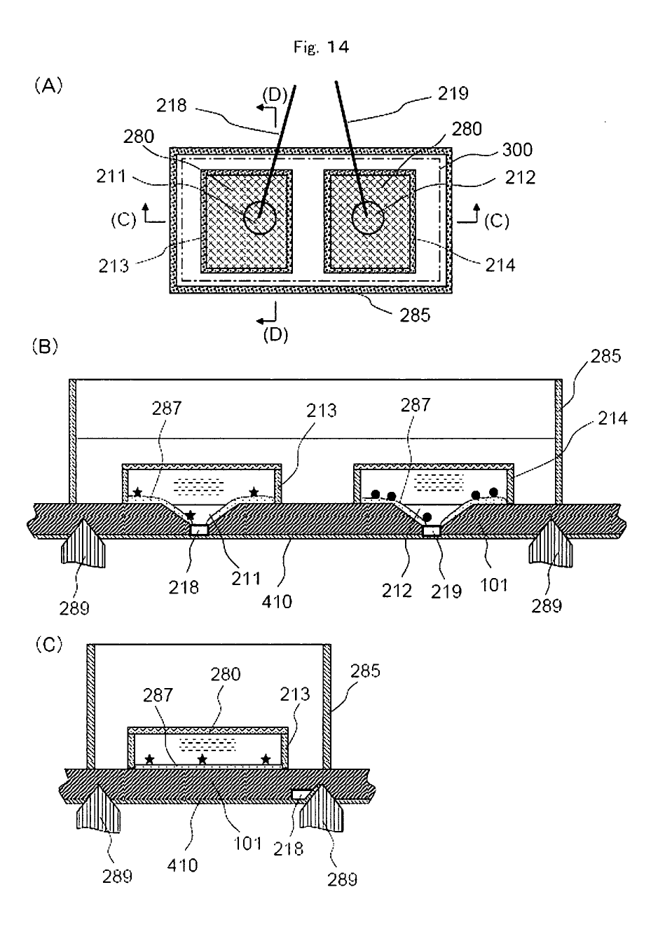

FIG. 14(A) through FIG. 14(C) show an example of processing for cutting the culturing tanks 213 and 214 together with the chip substrate 101 from the cell separation and culturing apparatus 100. FIG. 14(A) is a plan view showing only the culturing tanks 213 and 214 of the cell separation and culturing apparatus 100 and the reservoir 285 surrounding the culturing tanks 213 and 214. One-dot chain line 300 represents the cutting line along which the culturing tanks 213 and 214 are cut off together with the chip substrate 101. FIG. 14(B) is a cross-sectional view taken along line C-C of FIG. 14(A) and seen in the direction of the arrows thereof during the cutting operation. FIG. 14(C) is a cross-sectional view taken along line D-D of FIG. 14(A) and seen in the direction of arrows thereof during the cutting operation.

Before cutting off the culturing tanks 213 and 214, it is preferable that the buffer solution (medium) in the reservoir 285 is removed. In this case, the buffer solution (medium) in the culturing tanks 213 and 214 is in the same state as that after the separation operation is finished; i.e., the culturing tanks 213 and 214 accommodates the separated and collected cells and also the buffer solution (medium). Reference numeral 289 represents cutting teeth, which are formed such that the cutting tips thereof match the cutting line represented with the one-dot chain line in FIG. 14(A). The cutting tip of each cutting tooth 289 is formed to have a relatively large angle of about 60° to 130°. This is because the chip substrate 101 is cut as if being crushed with the cutting teeth 289 heated to about 80° C. to 110° C. In other words, the operation of cutting off the culturing tanks 213 and 214 also needs to close the flow channels 218 and 219 in communication with the culturing tanks 213 and 214. As shown in FIG. 14(C) which is the cross-sectional view taken along line D-D in FIG. 14(A) and seen in the direction of the arrows thereof, the flow channel 218 appears to have a lengthy opening (on the right). When the tip of the cutting tooth 289 is pushed to the chip substrate 101 at this position, the laminate film 410 is pushed up along the slanted face of the tip of the cutting tooth 289 (the right cutting tooth 289 in FIG. 14(C)) and thus closes the flow channel 218. In a portion with no opening such as the flow channel 218 or 219, the chip substrate 101 is cut off by the tips of the cutting teeth 289 as shown in FIG. 14(B) and in the left part of FIG. 14(C).

(Example of Optical System)

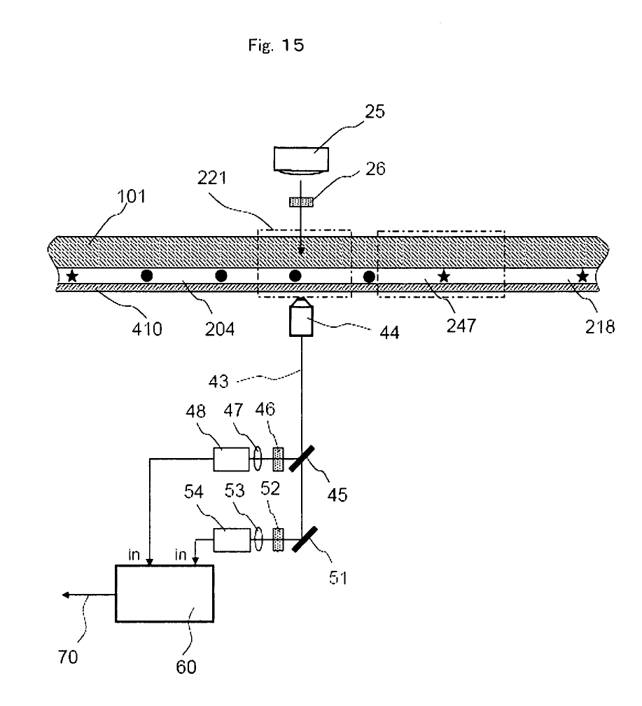

FIG. 15 is an overall conceptual view of an optical system in the cell detection area 221. A light source 25 for radiating light to a cell, and a filter 26, are provided on the top surface of the chip substrate 101. On the bottom surface of the chip substrate 101, a detection system for detecting light radiated to the cell is provided. FIG. 15 shows a cross-sectional view from the cell detection area 221 through the cell separation area 222 to the flow channel 218, along the flow direction of the flow channel 218 as shown in FIG. 2. The cells are modified with a fluorescent dye, gold particles or nanoparticles as shown in FIG. 10.

The cell flowing in the flow channel 218 in the cell detection area 221 is irradiated with light from the light source 25 through the filter 26. An image of the cell irradiated with the light is detected by an objective lens 44, and is captured as an image by a CCD camera 48 via a dichroic mirror 45, a filter 46, and a lens 47. The image data obtained by the CCD camera 48 is transferred to a computer 60 having an image processing function. The image data of the cell is checked against the prepared image data on the cell to be detected. When determining that the image data obtained by the CCD camera 48 has a predetermined relationship with the prepared image data on the cell to be detected, the computer 60 outputs a signal 70 to turn on the switch 216 of the cell separation and culturing apparatus 100. Thus, in the cell separation area 222, a voltage is applied to the buffer solution flowing in the flow channel 247 obtained by merging the microscopic flow channel 240 and the microscopic flow channel 204′, and thus a force is caused to act on the cell. Needless to say, the moving velocity of the cell flowing down the flow channel (the flow rate of the buffer solution in the flow channel 247) is separately detected, so that the voltage is applied at the timing when the cell evaluated in the cell detection area 221 passes the cell separation area 222.

When the cell is modified with a fluorescent dye, the fluorescence at the cell irradiated with the light is detected by the objective lens 44, passes through the dichroic mirror 45, and is captured by a photomultiplier 54 as a light spot via a reflective mirror 51, a fluorescent filter 52 and a lens 53. Alternatively, when the cell is modified with gold microparticles or nanoparticles, the scattering light from the cell irradiated with the laser light is detected by the objective lens 44. In this case, the fluorescent filter 52 is removed. The light detected by the objective lens 44 passes the dichroic mirror 45, and captured by the photomultiplier 54 as a light spot via the reflective mirror 51 and the lens 53. The light spot obtained by the photomultiplier 54 is sent to the computer 60 having a light processing function, and the computer 60 determines whether the cell has been modified as predetermined. When determining that the light spot is from the cell which has been modified as predetermined, the computer 60 outputs a signal 70 to turn on the switch 216 of the cell separation and culturing apparatus 100. Thus, in the cell separation area 222, a voltage is applied to the buffer solution flowing in the flow channel 247 obtained by merging the microscopic flow channel 240 and the microscopic flow channel 204′, and a force is caused to act on the cell. Needless to say, the moving velocity of the cell flowing down the flow channel (the flow rate of the buffer solution in the flow channel 247) is separately detected, so that the voltage is applied at the timing when the cell evaluated in the cell detection area 221 passes the cell separation area 222.

In the case where the CCD camera 48 is a photon counter or a photomultiplier, the intensity of the scattering light from the cell or the gold microparticles or nanoparticles bound to the cell may be continuously measured and sent to the computer 60 in accordance with the intensity change. The computer 60, having the light processing function, determines whether or not the cell has been modified as predetermined. In the case where the photomultiplier 54 is an optoelectric double speed camera, which area of the cell is labeled with fluorescence may be captured by images, and such information may be sent to the computer 60. The computer 60, having the light processing function, checks the obtained image data against the prepared image data on the cell to be detected. In this manner, it can be determined whether or not the cell has been modified as predetermined more precisely.

Needless to say, the image processing and the fluorescence or scattering light processing may be used together. The image data captured by the camera 48 may be displayed on a computer monitor such that the user can observe the cell.

What is claimed is:

1. A cell separation and culturing chip, comprising:
a substrate;
a flow channel formed in one surface of the substrate for allowing a cell-containing buffer solution to flow down;
a cell information detection area which is a predetermined area in the flow channel for detecting information on a cell flowing down the flow channel;
a cell separation area for allowing the cell to flow to any one of a plurality of flow channel branches in communication with the flow channel, in accordance with the information on the cell detected downstream with respect to the cell information detection area; and
a plurality of culturing tanks provided downstream with respect to the plurality of flow channel branches, each for keeping the cell-containing buffer solution which has flown down the respective flow channel branch;
wherein the culturing tank provided downstream with respect to the flow channel branch, to which a cell corresponding to the information detected in the cell information detection area and fulfilling a predetermined condition is caused to flow, is covered with a semipermeable membrane at a top surface thereof for preventing bacteria or the like from entering the culturing tank.

2. A cell separation and culturing chip according to claim 1, wherein the substrate is a plastic substrate formed by injection molding using a mold, and the flow channel is formed of a groove formed in the one surface of the plastic substrate and a laminate film for covering the groove.

3. A cell separation and culturing chip according to claim 1, wherein the information on the cell detected in the cell information detection area is obtained from image information on the cell.

4. A cell separation and culturing chip according to claim 1, wherein the cell flowing down the flow channel together with the buffer solution is modified with a predetermined fluorescent material via an aptamer, and the information on the cell detected in the cell information detection area is obtained from fluorescent luminance information provided by the fluorescent material modifying the cell.

5. A cell separation and culturing chip according to claim 1, wherein the cell flowing down the flow channel together with the buffer solution is modified with predetermined gold particles or nanoparticles via an aptamer, and the information on the cell detected in the cell information detection area is obtained from scattering light information provided by the gold particles or nanoparticles modifying the cell.

6. A cell separation and culturing chip according to claim 1, wherein the cell separation area has openings for a plurality of gel electrodes formed of an electrolyte-containing gel, the plurality of openings being provided as opposing to each other on both sides of the flow channel in which the cell flows down together with the buffer solution and being located offset from each other with respect to the flow of the buffer solution, and the cell is sent to one of the plurality of flow channel branches by the cell separation area in accordance with whether a predetermined electric current flows or not between the plurality of gel electrodes in the cell separation area.

7. A cell separation and culturing chip according to claim 4, wherein the semipermeable membrane has a hole through which ribozyme for decomposing the aptamer can pass.

8. A cell separation and culturing chip according to claim 1, wherein an inner bottom surface of each of the culturing tanks is covered with a layer of collagen, polylysine or fibronectin applied thereto, or is treated to be hydrophobic.

9. A cell culturing method comprising the steps of:
collecting a cell in a culturing tank in a cell separation and culturing chip comprising: a substrate; a flow channel formed in one surface of the substrate for allowing a cell-containing buffer solution to flow down; a cell information detection area which is a predetermined area in the flow channel for detecting information on a cell flowing down the flow channel; a cell separation area for allowing the cell to flow to any one of a plurality of flow channel branches in communication with the flow channel, in accordance with the information on the cell detected downstream with respect to the cell information detection area; and a plurality of culturing tanks provided downstream with respect to the plurality of flow channel branches, each for keeping the cell-containing buffer solution which has flown down the respective flow channel branch; wherein the culturing tank provided downstream with respect to the flow channel branch, to which a cell corresponding to the information detected in the cell information detection area and fulfilling a predetermined condition is caused to flow, is covered with a semipermeable membrane at a top surface thereof for preventing bacteria or the like from entering the culturing tank; said cell to be collected is the cell corresponding to the information fulfilling the predetermined condition, and said culturing tank in which said cell is to be collected is the culturing tank to which the cell corresponding to the information fulfilling the predetermined condition is caused to flow;
after the cell corresponding to the information fulfilling the predetermined condition is collected in the culturing tank, closing the flow channel branch in communication with the culturing tank and separating the culturing tank from the cell separation and culturing chip; and
putting the separated culturing tank into a culturing device containing a predetermined medium, and culturing the cell collected in the culturing tank.

10. A cell culturing method according to claim 9, wherein the separation of the culturing tank from the cell separation and culturing chip is performed by thermally separating the culturing tank together with an area of the substrate having the culturing tank therein.

11. A cell culturing method according to claim 9, wherein an inner bottom surface of each of the culturing tanks is covered with a layer of collagen, polylysine or fibronectin applied thereto, or is treated to be hydrophobic.
